(19) United States
(12) Patent Application Publication
(10) Pub. No.: US 2026/0053426 A1
(43) Pub. Date: Feb. 26, 2026

(54) METHOD, PROGRAM, AND APPARATUS FOR DETECTING SMALL INTESTINAL BACTERIAL OVERGROWTH

(71) Applicant: Atmo Biosciences Limited, Box Hill (AU)

(72) Inventors: James JOHN, Box Hill (AU); Malcolm HEBBLEWHITE, Box Hill (AU); Kyle BEREAN, Box Hill (AU); Adam CHRIMES, Box Hill (AU)

(21) Appl. No.: 19/105,649

(22) PCT Filed: Aug. 22, 2023
(86) PCT No.: PCT/AU2023/050804

§ 371 Date: Feb. 21, 2025

(30) Foreign Application Priority Data
Aug. 23, 2022 (AU) 2022902415
Apr. 6, 2023 (AU) 2023901021

(51) Int. Cl. A61B 5/00; A61B 5/01; A61B 5/07; G16H 15/00

(52) U.S. Cl. CPC A61B 5/4255; A61B 5/0084; A61B 5/01; A61B 5/073; A61B 5/6861; G16H 15/00; A61B 2562/0271; A61B 2562/029

(57) ABSTRACT

Embodiments include a method for detecting small intestinal bacterial overgrowth, SIBO, the method comprising: obtaining data representing a time series of readings from gas sensor hardware housed within an ingestible capsule device orally ingested by a subject, identifying the data corresponding to timing of passage through the small intestine, and determining whether or not the data indicates presence of SIBO.

### FIELD

This invention lies in the field of medicine and healthcare and in particular to digestive and gastrointestinal health. The invention specifically relates to detection, diagnosis, and/or measurement of small intestinal bacterial overgrowth (SIBO).

### BACKGROUND

Accurate diagnosis of small intestinal bacterial overgrowth (SIBO) has been a challenge for clinicians and researchers. The accepted standard for SIBO diagnosis is jejunal aspirate, which involves endoscopic retrieval of a fluid sample from the proximal end of the small bowel and subsequent analysis of bacterial content. Aspirate is prone to inaccuracies—false negatives are common as endoscopic tubes cannot reach distal areas of the small bowel, and false positives can occur due to contamination of the sample by flora native to the mouth or oesophagus (Ghoshal et al., 2011). The invasive nature of the jejunal aspirate test is unpleasant for patients.

An alternative diagnostic tool currently in use is the breath test, which measures the H2 or CH4 percentage in the breath. Breath testing has low sensitivity, around 40%, however is less invasive than aspirate and so is more commonly used (Paterson et al. 2017). Massey et al. (2021) has suggested that both diagnostic tools lack specificity for SIBO, instead differentiating only between healthy and unhealthy states.

A further complication is that SIBO is commonly a comorbidity for other gastrointestinal diseases, including Irritable Bowel Syndrome (IBS), which could affect the specificity of diagnostics. A positive outcome of this is that many papers have investigated the co-incidence of IBS and SIBO and, while reported prevalence of SIBO in IBS patients can vary widely between studies, this provides a large data set linking SIBO to a more easily diagnosed GI disease. It is desirable to develop a SIBO diagnostic method that compares favourably in terms of prevalence among IBS patients to prevalence among IBS patients in the literature.

### REFERENCES

Ghoshal, U. How to Interpret Hydrogen Breath tests. J Neurogastroenterol Motil. 2011 July; 17(3);
Paterson, W., Camilleri, M., Simren, M., Boeckxstaens, G., Vanner, S. Breath Testing Consensus Guidelines for SIBO: RES IPSA LOCQUITOR. Journal of Gastroenterology. 2017 December
Massey. B., Wald, A. Small Intestinal Bacterial Overgrowth Syndrome: A Guide for the Appropriate Use of Breath testing. Digestive Diseases and Sciences. 2021; 66:338-347.

### SUMMARY

Embodiments include a method for detecting presence or absence of small-intestinal bacterial overgrowth, SIBO, in a subject, the method comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of composition of the gas mixture at the location of the ingestible capsule device in the gastrointestinal tract of the subject; using the gas sensor data to calculate a metric representing fluctuation of the gas sensor data during a passage of the ingestible capsule device through the small intestine of the gastrointestinal tract; determining presence or absence of SIBO in the subject at least partially in dependence upon the metric representing fluctuation.

The metric representing fluctuation may be an aggregation of cumulative fluctuation between two times.

The metric representing fluctuation may be a statistical measure such as variance or standard deviation.

The fluctuation may be, for example, the irregular or residual component after a trend has been removed, subtracted, or otherwise compensated for. For example, a trend may be represented by a trend line (such as a first order polynomial), and fluctuations are deviations from the trend line. The metric representing fluctuation may be, for example, a summation or aggregate fluctuation. Noting that the summation or aggregation is of the amount of fluctuation as a scalar so that fluctuations to the positive and negative side of the reference or trend line accumulate rather than cancelling out. Alternative metrics include a number of instances in which the gas sensor data is at a value more than a threshold distance from a reference or trend line, distance being a vertical distance i.e. a deviation or difference in magnitude. Aggregate fluctuation may be calculated by integrating or otherwise summing area between trend line and a line joining time series data points to one another, or by otherwise summing or accumulating distance between each time series data point and the trend line. Trend line may be a single-order polynomial. In summary, fluctuation is the tendency to deviate from a trend line. In physical terms, aggregate fluctuation represents unevenness in rate of production of fermentation gases, and would be caused by distinct clumps or populations of fermentation-causing bacteria in the small intestine.

The trend line may be a time representation or a displacement representation of the capsule in the small intestine. For example, if the capsule were to be stationary for a period of time the trend line (of displacement) may have a flat region at that period.

Aggregate refers to the summation of the fluctuation over the period in question, which period is a period during which the capsule is in the small intestine. Said period may be determined on-capsule on-the-fly, i.e. in more or less real-time by processing data captured on the capsule and identifying an indicator or indicators in the data of entry into, or exit from, the small intestine. Alternatively the period may be determined retrospectively in post-processing of data captured by the capsule.

The gas or gases may be one or both of carbon dioxide CO2, hydrogen H2. The gas or gases may further comprise methane. The gas or gases may include one or more VOCs.

Alternatively, methods comprise a method for detecting presence or absence of small-intestinal bacterial overgrowth, SIBO, in a subject, the method comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, each reading representing a composition of the gas mixture at the location of the ingestible capsule device in the gastrointestinal tract of the subject; using the gas sensor data to calculate a gradient of a best fit line (single-order polynomial) fitted to the gas sensor data from passage of the ingestible capsule device through the small intestine of the gastrointestinal tract; and determining presence or absence of SIBO in the subject at least partially in dependence upon the gradient of the best fit line.

Breath tests are inaccurate because of the indirect measurement method where the by-products of fermentation needs to be absorbed into the blood stream, before being expelled from the lungs. It is also suspected that false positive results can be caused by the test substrate reaching the large bowel earlier than expected due to variability in patient's transit times. This can be erroneously interpreted as early fermentation in the small bowel indicating SIBO. Embodiments provide a direct measurement technique which addresses the inaccuracies in the indirect measurement methods.

The below optional features may be combined with either of the above method based on calculating the metric representing fluctuation and determining presence or absence of SIBO at least partially in dependence on the calculated metric representing fluctuation, or the above method based on calculating the gradient of the best fit line and determining presence or absence of SIBO at least partially in dependence on the calculated gradient.

Optionally, embodiments further comprise, based on the determined metric representing fluctuation, measuring a level of fermentation activity detected in the small bowel of the subject, and including the measured level in the generated report.

Optionally, the determining comprises, as a first comparison, comparing the metric representing fluctuation with a predefined threshold, and using a result of the first comparison to determine presence or absence of SIBO, and wherein the metric representing fluctuation is an aggregate fluctuation.

Optionally, the gas sensor data is obtained by obtaining readings from an environmental temperature sensor housed within the ingestible capsule device representing environmental temperature at the ingestible capsule device, and compensating sampled values of an output signal generated by the gas sensor hardware to account for variations in environmental temperature, the gas sensor data being the compensated values.

Variations in environmental temperature in the small intestine would be caused by consumption of food & drink, and cause change in behaviour of gas sensors such as TCD gas sensor. Using a lookup table or a formula or some other processing technique, sampled values of a signal output by the gas sensor hardware can be corrected to remove or otherwise cancel the variation in values caused by variation in environmental temperature, so that the remaining variation in values is attributable to changes in fluid composition at the capsule.

Based on readings from an environmental temperature sensor housed within the ingestible capsule device and monitoring environmental temperature at the ingestible capsule device, compensating the gas sensor data for variations in environmental temperature; the concentration of the one or more gases is determined/measured/calculated from the compensated gas sensor data.

Embodiments include an apparatus comprising memory hardware and processor hardware, the memory hardware storing processing instructions which, when executed by the processor hardware, cause the processor hardware to perform a method of an embodiment.

Optionally, the gas sensor data represents a concentration of a specific gas or gases. Furthermore, the gas sensor data may be obtained by processing sampled values of an output signal generated by the gas sensor hardware to extract the concentration of the specific gas or gases. Furthermore, the specific gas or gases may be one or more from among: carbon dioxide CO2, hydrogen H2, methane, and one or more VOCs. Furthermore, determining presence or absence of SIBO in the subject may be at least partially in dependence upon the concentration of the specific gas or gases exceeding a predefined threshold concentration at one or a predefined threshold number of locations during passage of the ingestible capsule device through the small intestine.

Optionally, the gas sensor data is, or is in direct proportion to, sampled values of an output signal generated by the gas sensor hardware.

Optionally, the method further comprises: fitting a trend line to the gas sensor data; wherein the determining presence or absence of SIBO in the subject is at least partially in dependence upon the metric representing fluctuation and at least partially in dependence upon a gradient of the trend line or an average gradient of the trend line.

Optionally, the determining comprises, as a second comparison, comparing a gradient of the trend line with a second predefined threshold; combining a result of the first comparison the gradient with the result of the second comparison to detect presence or absence of small-intestinal bacterial overgrowth in the subject.

Optionally, the determining comprises calculating a weighted average or weighted sum of characteristics including at least the metric representing fluctuation and the trend line gradient, comparing the weighted average with a predefined threshold, and determining presence or absence of SIBO in dependence upon the result of the comparison. Furthermore, the gas sensor data may represent a concentration of a specific gas or gases, and the characteristics further comprise a number of times, or a duration for which, during passage of the capsule through the small intestine that the concentration of the specific gas or gases exceeds a predefined threshold concentration.

Optionally, the gas sensor hardware comprises a TCD gas sensor and the gas sensor data represents a time series of readings from the TCD gas sensor.

Optionally, the method further comprises: detecting a gas sensor data gastric-duodenal transition indicator among the gas sensor data and/or detecting a gas sensor data ileocecal junction transition indicator among the gas sensor data, and based on a timing of the detected gas sensor data gastric-duodenal transition indicator and/or the detected gas sensor data ileocecal junction transition indicator, determining timing of the passage of the ingestible capsule device through the small intestine of the subject.

Optionally, the method further comprises: obtaining accelerometer data representing a time series of readings from an accelerometer housed within the ingestible capsule device, the time series of readings being taken during the passage of the ingestible capsule device through the gastrointestinal tract of the subject; detecting an accelerometer data gastric-duodenal indicator and/or an accelerometer data ileocecal junction indicator in the accelerometer data, and determining the timing of the passage of the ingestible capsule device through the small intestine of the gastrointestinal tract based on the accelerometer data gastric-duodenal indicator and/or the accelerometer data ileocecal junction indicator.

Optionally, the method further comprises: obtaining reflectometer data representing a time series of readings from a reflectometer housed within the ingestible capsule device, the reflectometer comprising a transmission antenna connected in series with a directional coupler configured to measure a reflected signal from the transmission antenna, the time series of readings being taken during the passage of the ingestible capsule device through the gastrointestinal tract of the subject; detecting a reflectometer data gastric-duodenal indicator and/or a reflectometer data ileocecal junction indicator in the reflectometer data, and determining the timing of the passage of the ingestible capsule device through the small intestine of the gastrointestinal tract based on the reflectometer data gastric-duodenal indicator and/or the reflectometer ileocecal junction indicator. Furthermore, the determining the timing of the passage of the ingestible capsule device through the small intestine of the subject may comprise: determining a timing of a passage of the ingestible capsule device across the gastric-duodenal junction based on one or more from among: the gas sensor data gastric-duodenal indicator; the accelerometer data gastric-duodenal indicator; and the reflectometer data gastric-duodenal indicator. Determining the timing of the passage of the ingestible capsule device through the small intestine of the subject may comprise: determining a timing of a passage of the ingestible capsule device across the ileocecal junction based on one or more from among: the gas sensor data ileocecal junction indicator, the accelerometer data ileocecal junction indicator, and the reflectometer data ileocecal junction indicator.

Optionally, methods further comprise: quantifying an amount of small-intestinal bacterial overgrowth in the subject according to the value of the metric representing fluctuation, quantifying an amount of small-intestinal bacterial overgrowth in the subject according to the gradient of the trend line, or quantifying an amount of small-intestinal bacterial overgrowth in the subject according to a number of times, or a duration for which, during passage of the capsule through the small intestine that the concentration of the specific gas or gases exceeds a predefined threshold concentration.

Optionally, methods include generating a report including the detected presence or absence of small-intestinal bacterial overgrowth in the subject. Methods may further comprise: based on one or more from among: a detected fermentation indicator, the metric representing fluctuation, a number of events, or a duration for which, during passage of the capsule through the small intestine that a concentration of the specific gas or gases represented by the gas sensor data exceeds a predefined threshold concentration, and the gradient of a trend line fitted to the gas sensor data, measuring a level of fermentation activity detected in the small intestine of the subject, and including the measured level in the generated report. Methods may further comprise determining an estimated location or locations within the small intestine of fermentation activity, based on a timing of: deviations from a trend line contributing to a metric representing fluctuation, and/or a timing of events at which concentration of a specific gas or gases represented by the gas sensor data exceeds a predefined threshold concentration. The report may comprise the measured level of fermentation activity and/or the estimated location or locations within the small intestine of fermentation activity. Optionally, the method is performed by processor hardware and memory hardware within the ingestible capsule device, and the method further comprises wirelessly transmitting the report to a receiver device outside of the body of the subject. The wirelessly transmitting may be via a Bluetooth transceiver housed by the ingestible capsule device.

Optionally, the method is performed by a computing apparatus comprising processor hardware and memory hardware and receiving data directly or indirectly from the ingestible capsule device.

Optionally, part of the method is performed by processor hardware and memory hardware within the ingestible capsule device, the method further comprises wirelessly transmitting data representing the performed part of the method from a wireless transceiver housed by the ingestible capsule device to a receiver device outside of the body of the subject, and at the receiver device or a computing device in data communication therewith, receiving and using the transmitted data representing the performed part of the method to complete the method.

The result of the first comparison and/or the result of the second comparison may be considered to be fermentation indicators. A SIBO diagnosis may be contingent upon the detection of either or both of the fermentation indicators in the data obtained from the ingestible capsule device. Methods may include, in response to detecting the fermentation indicator, determining presence of SIBO in the subject and generating a report indicating the determined presence of SIBO in the subject.

Optionally the memory hardware and processor hardware are housed within the ingestible capsule device, the ingestible capsule device further comprises a wireless transmitter, and the processor hardware is configured to perform the method during passage of the ingestible capsule device through the gastrointestinal tract of the subject, and further to diagnosing SIBO in the subject, to transmit data indicating the diagnosis to a receiver device via the wireless transmitter.

Embodiments include an ingestible capsule device comprising: an ingestible indigestible bio-compatible housing; and, within the housing: a power source; sensor hardware including gas sensor hardware; processor hardware; memory hardware; and a wireless data transceiver, the memory hardware storing processing instructions which, when executed by the processor hardware, cause the processor hardware to perform a process comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of composition of the gas mixture at the location of the ingestible capsule device in the gastrointestinal tract of the subject; using the gas sensor data to calculate a metric representing fluctuation of the gas sensor data during a passage of the ingestible capsule device through the small intestine of the gastrointestinal tract; determining presence or absence of SIBO in the subject at least partially in dependence upon the metric representing fluctuation.

Embodiments include a computer program which, when executed by a processor, causes the processor to perform a method disclosed in the present summary or appended description, figures, and claims.

Advantageously, embodiments provide a reliable and accurate diagnosis method which relies upon data obtained by ingestion (and excretion) of an ingestible capsule device by a subject. The data obtained from the capsule enables accurate determination of presence or absence of SIBO in the patient. The inventors have identified a technique for detecting SIBO by determining a tendency to experience clumps or localised spots of high and low concentration of fermentation gases in the small intestine, this tendency being an indicator of SIBO. In addition, a general first-order polynomial trend line and in particular a gradient thereof provides a further indicator

In a particular example, the inventors have identified a signature in the signal output by a TCD gas sensor housed within an ingestible capsule device that is diagnostic of SIBO.

Embodiments include a method for detecting small intestinal bacterial overgrowth, SIBO, the method comprising: obtaining data representing a time series of readings from gas sensor hardware housed within an ingestible capsule device orally ingested by a subject, the time series of readings being taken during exposure of the gas sensor hardware to a gas mixture at the ingestible capsule device during passage of the ingestible capsule device through a gastrointestinal tract of the subject, each reading representing a composition of the gas mixture at the location of the ingestible capsule device in the gastrointestinal tract of the subject; detecting an ileocecal junction transition indicator among the obtained data, and based on a timing of the detected ileocecal junction transition indicator, detecting a fermentation indicator among the obtained data representing readings preceding the timing of the detected ileocecal junction indicator among the time series of readings; in response to detecting the fermentation indicator, determining presence of SIBO in the subject and generating a report indicating the determined presence of SIBO in the subject.

Optionally, the fermentation indicator is detected by processing the time series of readings from the gas sensor hardware to obtain a time series of values representing a concentration of a specific gas or gases within the gas mixture at the location if the ingestible capsule device, and detecting, as the fermentation indicator, the concentration of the specific gas or gases exceeding a predefined threshold.

Embodiments include a method for detecting small intestinal bacterial overgrowth, SIBO, the method comprising: obtaining data representing a time series of readings from gas sensor hardware housed within an ingestible capsule device orally ingested by a subject, identifying the data corresponding to timing of passage through the small intestine, and determining whether or not the data indicates presence of SIBO.

Embodiments include an ingestible capsule device comprising: an ingestible indigestible bio-compatible housing; and, within the housing: a power source; sensor hardware including gas sensor hardware; processor hardware; memory hardware; and a wireless data transmitter; the memory hardware storing processing instructions which, when executed by the processor hardware, cause the processor hardware to perform a method of an embodiment.

Embodiments include a computer program which, when executed by a processor, causes the processor to perform a process for detecting presence or absence of small-intestinal bacterial overgrowth, SIBO, in a subject, the process comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of composition of the gas mixture at the location of the ingestible capsule device in the gastrointestinal tract of the subject; using the gas sensor data to calculate a metric representing fluctuation of the gas sensor data during a passage of the ingestible capsule device through the small intestine of the gastrointestinal tract; determining presence or absence of SIBO in the subject at least partially in dependence upon the metric representing fluctuation. The computer program may be stored by a computer-readable medium. For example, the computer-readable medium may be a non-transitory computer-readable medium.

Embodiments include a method for detecting presence or absence of small-intestinal bacterial overgrowth, SIBO, in a subject, the method comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of a composition of the gas mixture at the location of the ingestible capsule device in the gastrointestinal tract of the subject; using the gas sensor data to calculate a gradient of a first order polynomial best fit line fitted to the gas sensor data from passage of the ingestible capsule device through the small intestine of the gastrointestinal tract; and determining presence or absence of SIBO in the subject at least partially in dependence upon the gradient of the best fit line.

Embodiments include a method for detecting presence or absence of small-intestinal bacterial overgrowth, SIBO, in a subject, the method comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of a composition of the gas mixture at the location of the ingestible capsule device in the gastrointestinal tract of the subject; wherein the gas sensor data represents a concentration of a specific gas or gases; the method further comprising determining presence or absence of SIBO in the subject at least partially in dependence upon the concentration of the specific gas or gases exceeding a predefined threshold concentration at one or a predefined threshold minimum number of locations during passage of the ingestible capsule device through the small intestine.

Optionally, the specific gas or gases is one or more from among hydrogen, carbon dioxide, and methane.

### DETAILED DESCRIPTION

Embodiments are described below, by way of example, with reference to the accompanying drawings, in which:

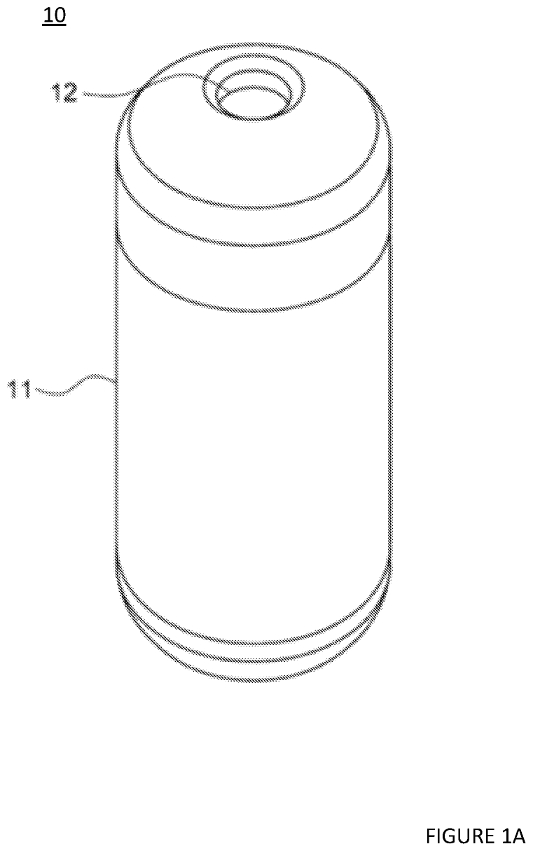

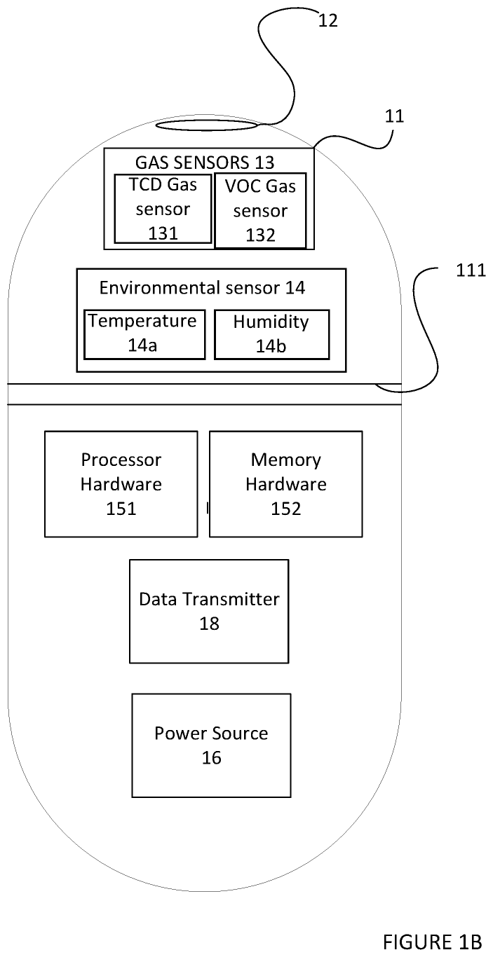

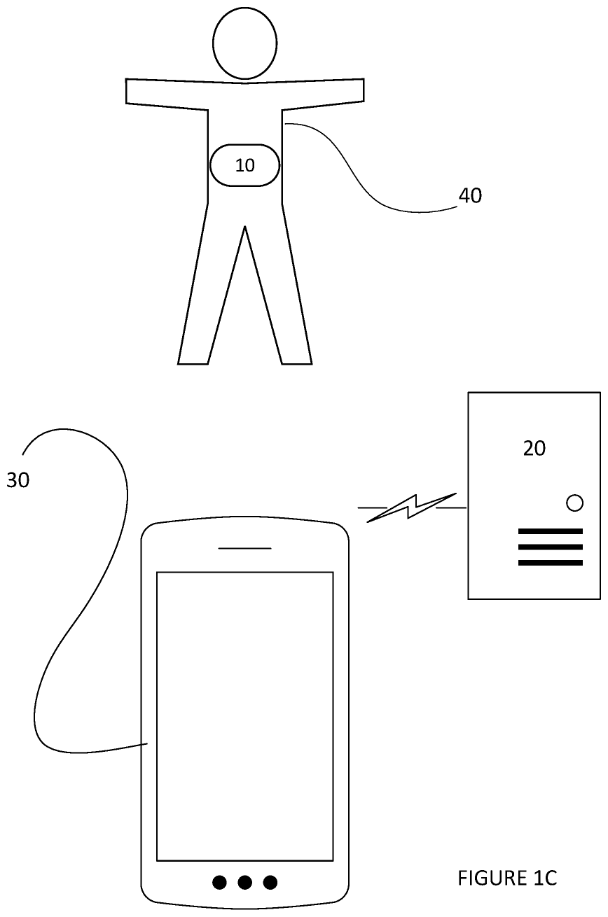

FIG. 1A illustrates an ingestible capsule device;

FIG. 1B is a schematic diagram of components of an ingestible capsule device;

FIG. 1C illustrates a system including an ingestible capsule device;

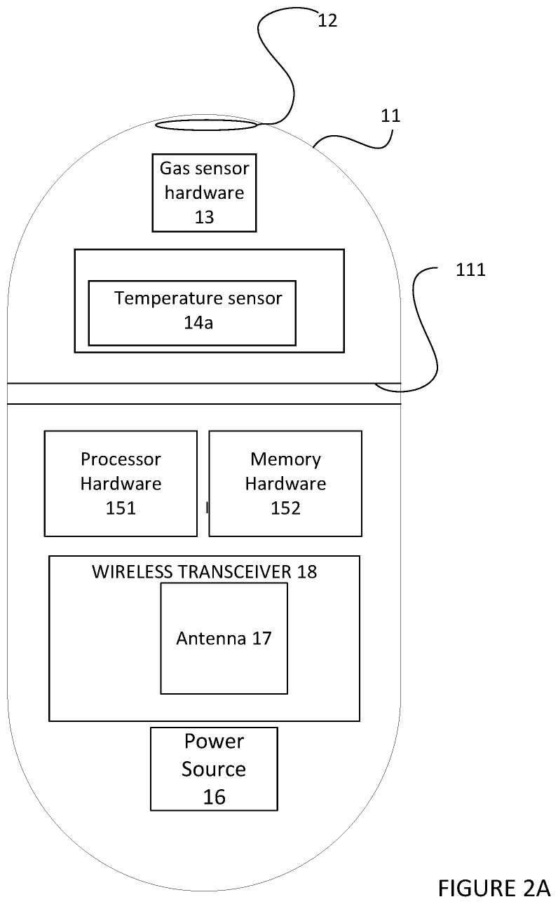

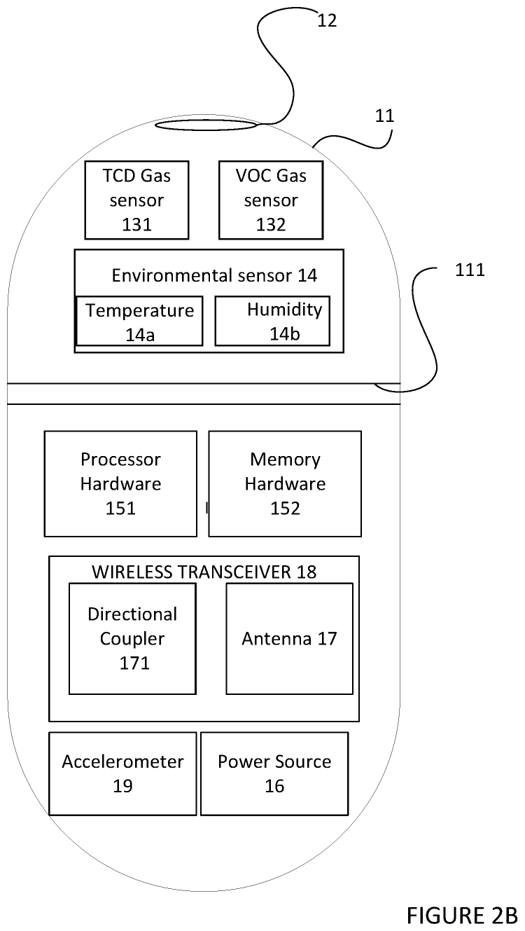

FIG. 2A is a schematic diagram of components of an ingestible capsule device;

FIG. 2B is a schematic diagram of components of an ingestible capsule device;

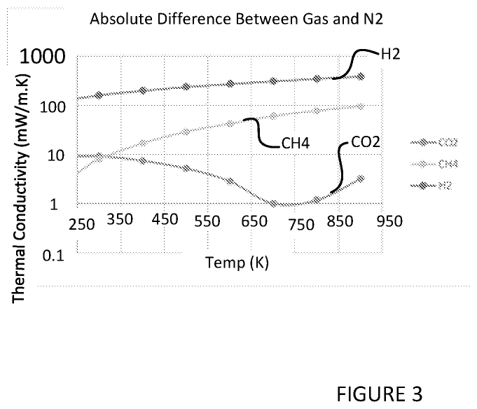

FIG. 3 illustrates changing sensitivity to constituent gases with operating temperature;

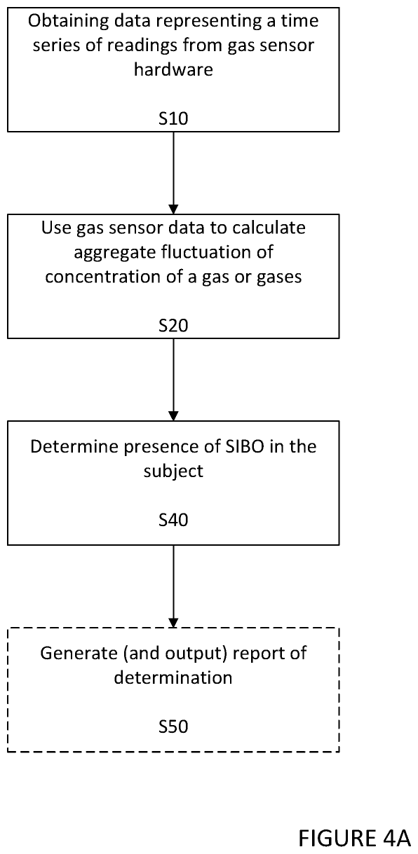

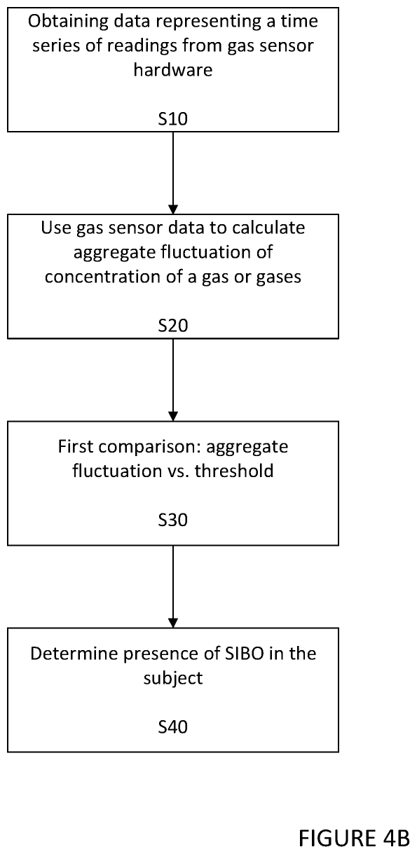

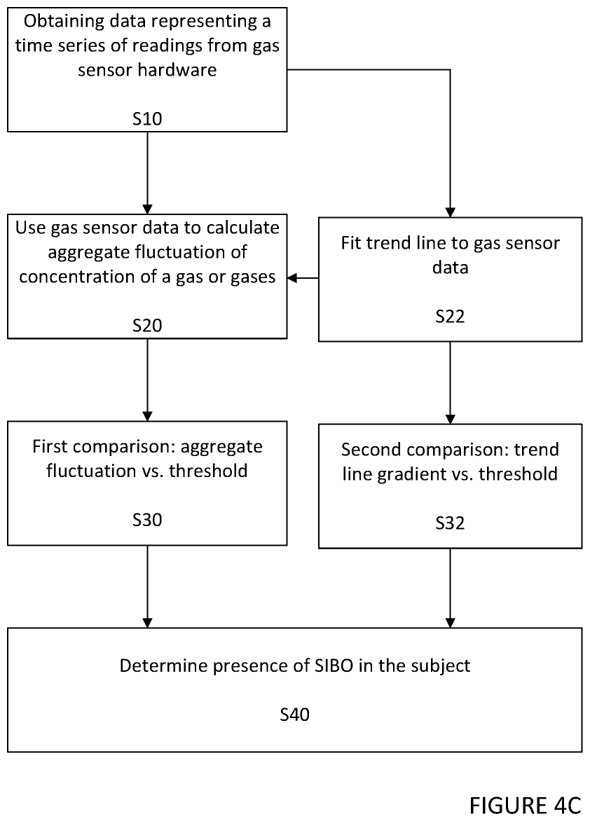

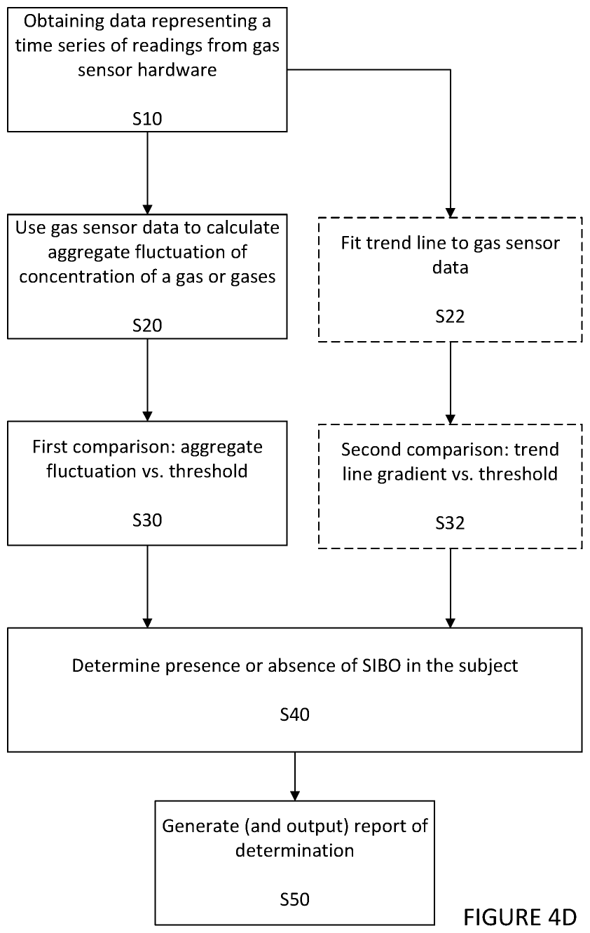

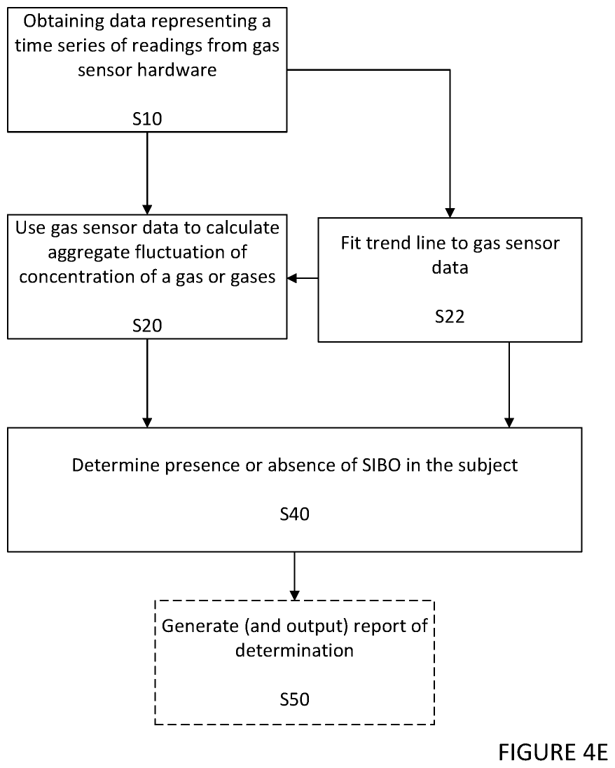

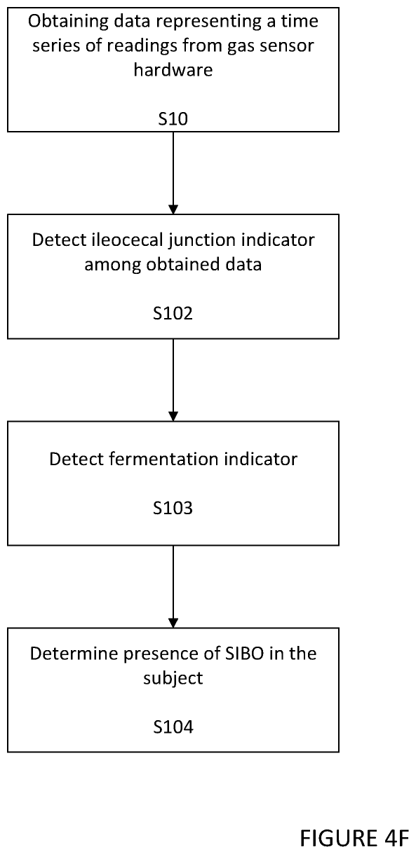

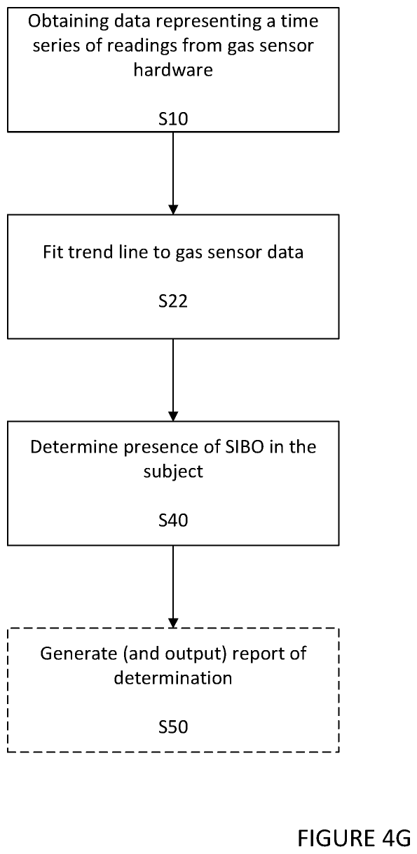

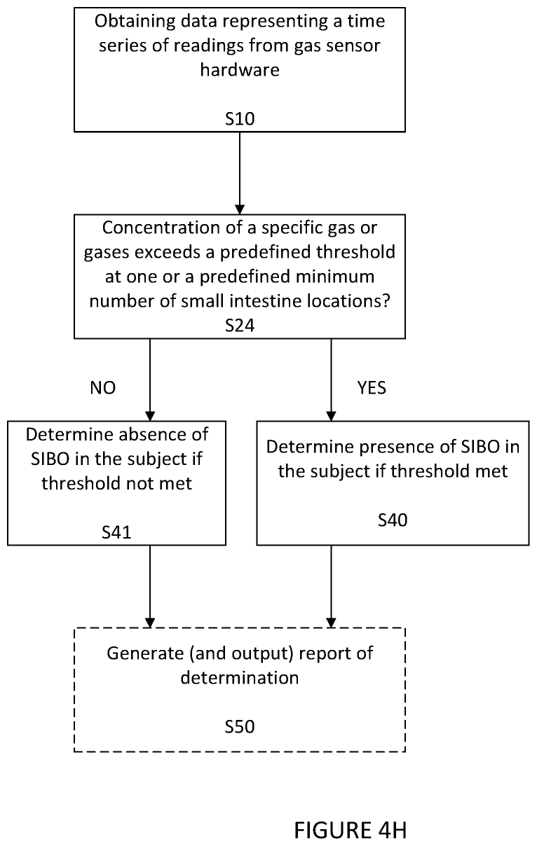

FIG. 4A illustrates a method;

FIG. 4B illustrates a method;

FIG. 4C illustrates a method;

FIG. 4D illustrates a method;

FIG. 4E illustrates a method;

FIG. 4F illustrates a method;

FIG. 4G illustrates a method;

FIG. 4H illustrates a method;

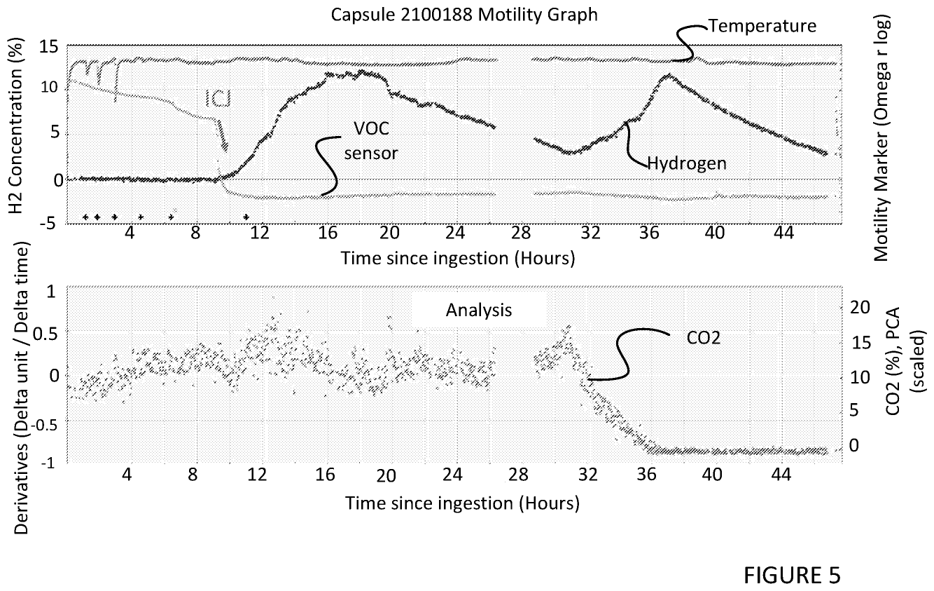

FIG. 5 illustrates a plot of data generated by an ingestible capsule device;

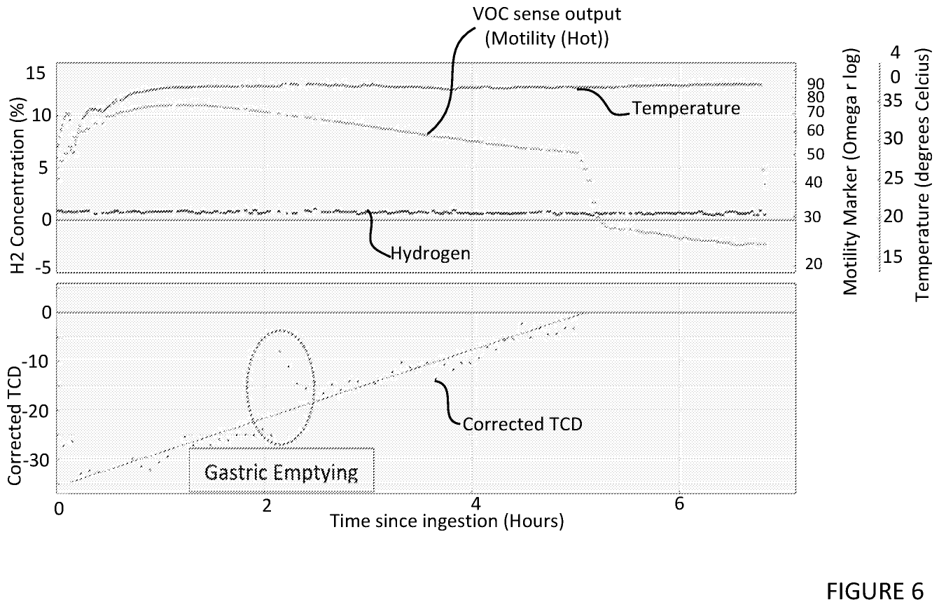

FIG. 6 illustrates a plot of data generated by an ingestible capsule device;

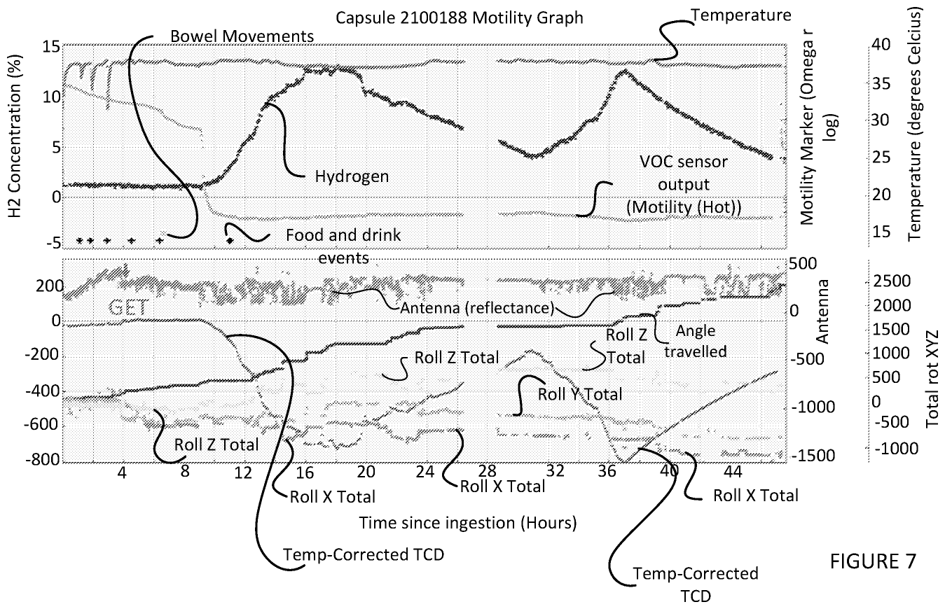

FIG. 7 illustrates a plot of data generated by an ingestible capsule device;

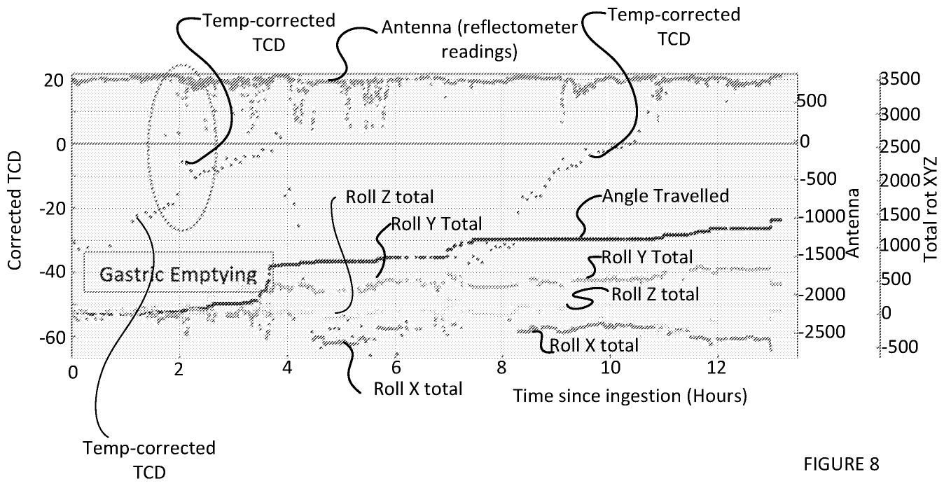

FIG. 8 illustrates a plot of data generated by an ingestible capsule device;

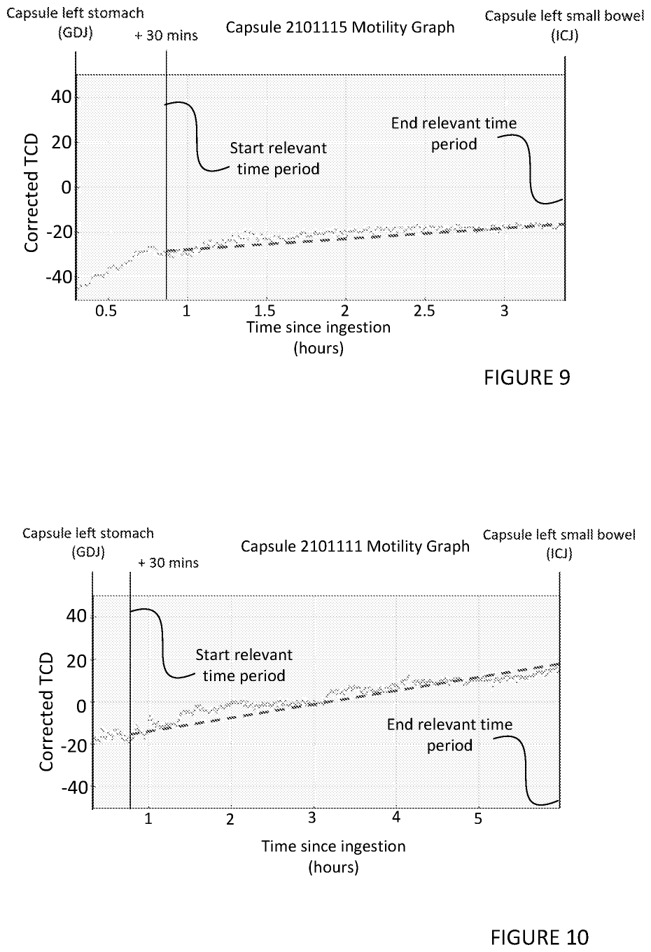

FIGS. 9 and 10 each illustrates a time series of readings from gas sensor hardware;

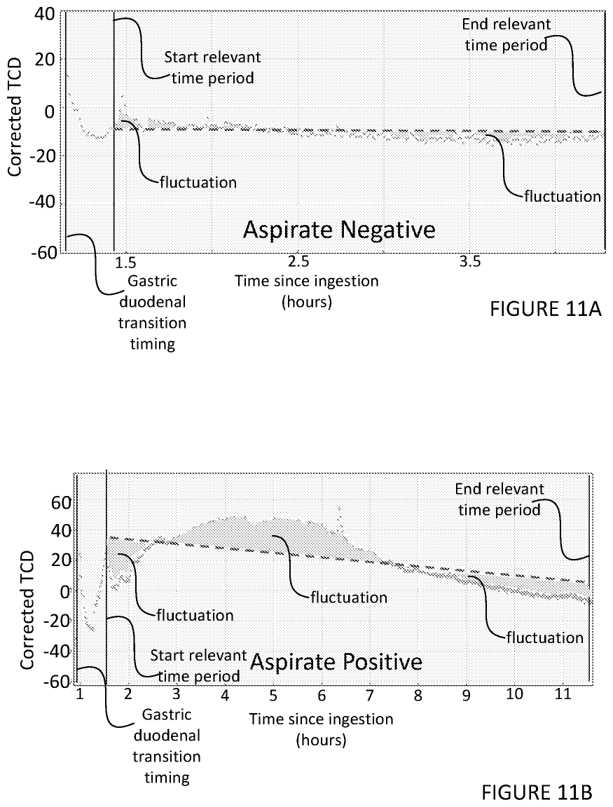

FIGS. 11A and 11B each illustrates a time series of readings from gas sensor hardware;

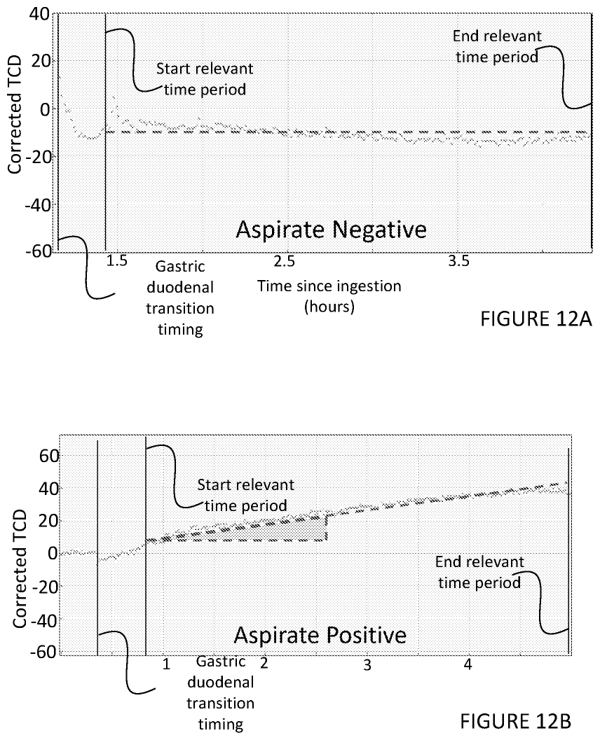

FIGS. 12A and 12B each illustrates a time series of readings from gas sensor hardware;

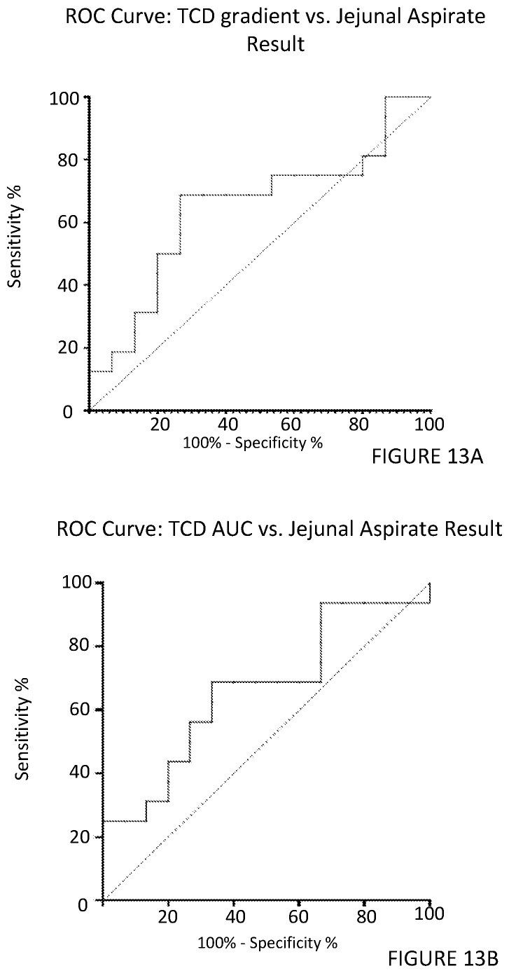

FIGS. 13A and 13B illustrate receiver operating characteristic curves for trial results;

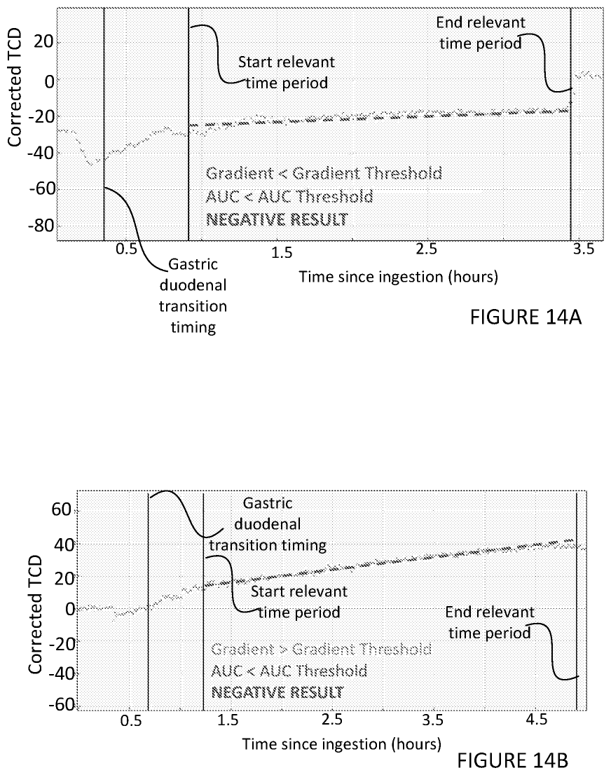

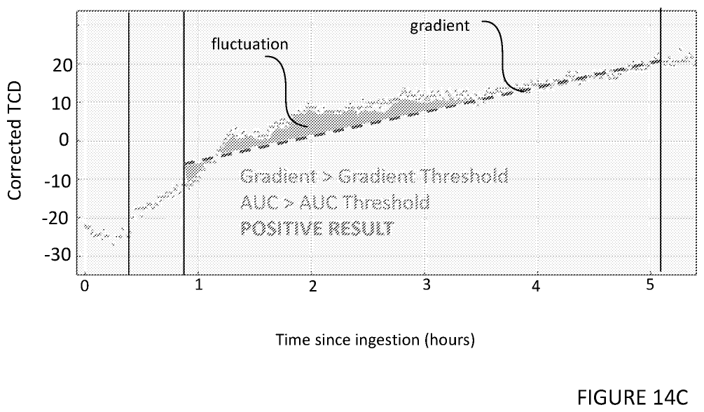

FIGS. 14A to 14C each illustrates a time series of readings from gas sensor hardware;

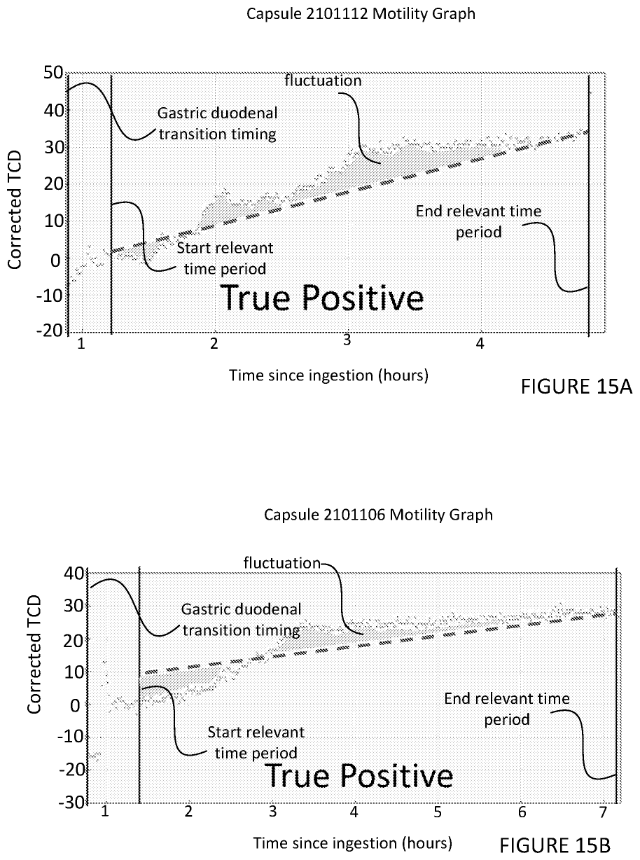

FIGS. 15A and 15B each illustrates a time series of readings from gas sensor hardware;

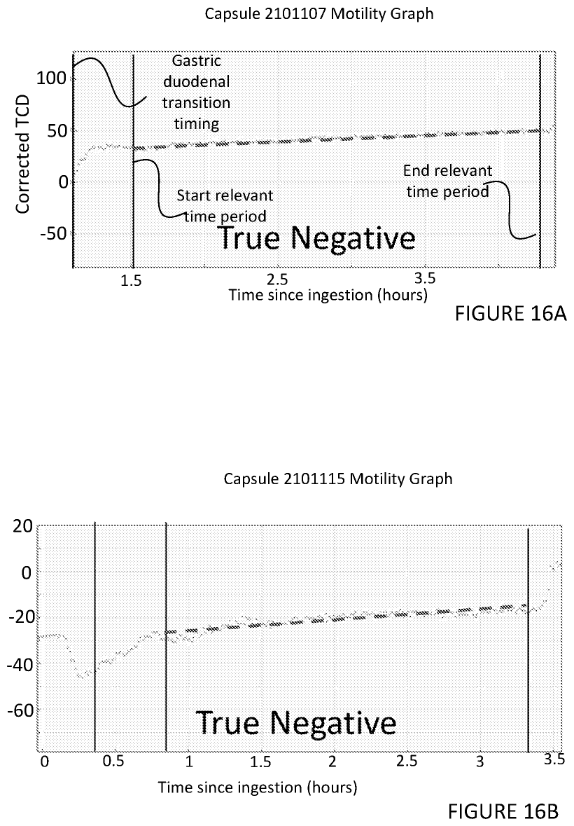

FIGS. 16A and 16B each illustrates a time series of readings from gas sensor hardware;

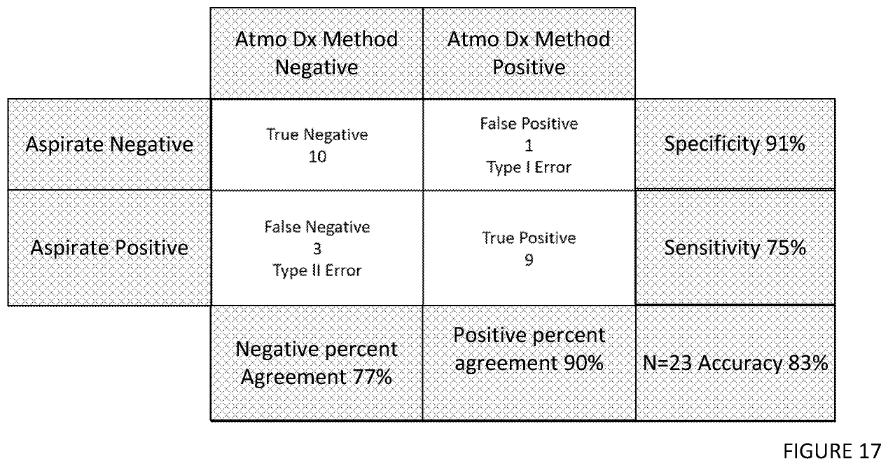

FIG. 17 presents results of comparison between a present method and jejunal aspirate testing; and

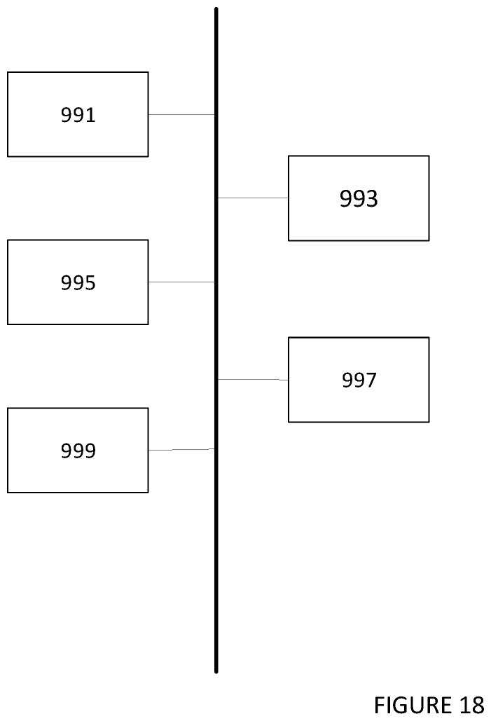

FIG. 18 illustrates a hardware arrangement of an apparatus.

### Ingestible Capsule Overview

FIGS. 1A, 1B, 2A, and 2B illustrate an ingestible capsule device 10, also referred to as an ingestible capsule or simply as a capsule. A system including the ingestible capsule device 10 is illustrated in FIG. 1C, during a live phase of the ingestible capsule device 10 (i.e. while the ingestible capsule device 10 is obtaining readings from within the GI tract of a subject mammal 40). Capsule housing is an ingestible indigestible bio-compatible material.

As shown in FIGS. 1A, 1B, 2A, and 2B the ingestible capsule device 10 consists of a housing such as a gas impermeable shell 11 which has an opening covered by a gas permeable membrane 12. A membrane 111 separates an exposed interior cavity exposed to the environmental gases entering the capsule 10 through the membrane 12 from a sealed-off interior cavity that is not exposed to the environmental gases. It is noted that circuitry carrying data and/or power may pass through the interior membrane 111 without sacrificing the integrity of the seal.

As shown in FIG. 1C, the system, in addition to the capsule, further comprises a receiver apparatus 30 which receives data transmitted by the capsule from within the GI tract of the subject mammal during the live phase. Concurrently or subsequently, the receiver apparatus 30 processes the received data and may also upload some or all of the received data to a remote processing apparatus 20 such as a cloud-based service for further processing. The remote computer 20 may be a cloud resource, or may be a standalone computer at a clinician premise at which the subject is a patient, or may be a server (be it cloud-based or otherwise) at a service provider to which the clinician is a subscriber/customer/servicer user.

Optionally, a system may further comprise a remote processing apparatus 20 such as a server forming part of a cloud computing environment or some other distributed processing environment. The remote processing apparatus 20 may be a server provided by or on behalf of a clinical centre at which subject 40 is a patient and taking responsibility for interpreting the results generated by the capsule 10 (i.e. the data transmission payload) and reporting them to the subject 40.

Data transmission payload is a term to refer to the payload of data transmitted away from the capsule 10 (that is, data representing the readings of the on-board sensor hardware, or reports or other results resulting from the on-board processing of the readings).

Connectivity between the capsule 10 and the receiver apparatus 30 is via the data transceiver 18 on the capsule, which may be part of a wireless transceiver, for example a Bluetooth transceiver, which may operate according to a standard Bluetooth transmission protocol or according to Bluetooth Long Range transmission protocol. Other operable communication technologies include LoRa, wifi and 433 MHz radio.

Internally the capsule 10 includes gas sensor hardware 13, which may be a TCD gas sensor 131, or a VOC gas sensor 132, or another type of gas sensor sensitive to changing concentrations of one or more gases associated with fermentation in the small intestine such as H2 or CO2. The capsule 10 may comprise a temperature sensor 14a, for sensing the temperature of the environment in which the capsule 10 resides. Optionally, the capsule 10 may further comprise a humidity sensor 14b. The capsule 10 comprises processor hardware 151 and memory hardware 152, which may be separate components or may both be provided on the same single chip. The processor hardware 151 and memory hardware 152 may be a microcontroller. The processor hardware 151 may be a microprocessor. The memory hardware 152 may be a non-volatile memory and the data stored thereon is accessible by the processor hardware 151. The processor hardware 151 processes data from signals received from the gas sensor hardware 13 and the temperature sensor 14a (and optionally also the reflectometer and accelerometer 19) and stores the processed data on the memory hardware 152. The processed data, or a portion thereof, is stored on the memory hardware 152 as a data transmission payload ready for transmission to a receiver apparatus 30 by the data transmitter 18. The processed data may be the readings from the sensor hardware (a collective term to refer to whichever are included on the capsule 10 from among gas sensor hardware 13, temperature sensor 14a, reflectometer, and accelerometer 19) may be the readings themselves (for example for a process of determining presence or absence of SIBO to be carried out at the receiver apparatus 30 or remote computing device 20) or may be a report or some other data representing a result of the determination of presence or absence of SIBO that has been carried out on the capsule 10.

The TCD gas sensor 131 may be a low temperature TCD gas sensor. The sensitivity of the TCD gas sensor may be <1% volume concentration sensitive. In the small intestine, the TCD gas sensor 131 senses carbon dioxide CO2 and hydrogen H2.

By way of example, the capsule illustrated in FIG. 2B houses, as sensor hardware, an environmental sensor 14 in the form of a temperature sensor 14a and/or a humidity sensor 14b, gas sensors in the form of a TCD gas sensor 131 and a VOC gas sensor 132, an accelerometer 19, and a reflectometer. Embodiments may include any single or combination of those individual sensors. Alternatively or additionally, embodiments may include one or more sensors not illustrated in FIG. 2B such as a spectrophotometer, Surface Acoustic Wave sensor, and/or Bulk Acoustic Resonator Arrays.

Ingestible capsule device 10 may include an environmental sensor 14 which may be an environmental temperature sensor 14a or may be an environmental temperature sensor 14a and a humidity sensor 14b. The gas sensor hardware 13 may be a TCD gas sensor 131, a VOC gas sensor 132, or a TCD gas sensor 131 and a VOC gas sensor 132, or another type of gas sensor sensitive to changing concentrations of gases associated with fermentation in the small intestine. As illustrated, the internal electronics also include a power source 16, for example, silver oxide batteries. The internal electronics also include a wireless transceiver 18 including an antenna 17. The internal electronics may also include a reed switch or some other mechanism for waking up the ingestible capsule device 10 upon removal from packaging. Other options for keeping the device switched off (or otherwise not consuming power) during storage include a physical switch pressed via a flexible part of the housing, or a photodetector and coupled field effect transistor that latches the microcontroller on when exposed to light. Or, for example, an NFC transceiver that responds to a signal transmitted from the receiver device 30, for example as triggered by an app configured to manage storing, processing, and exchange of data between the ingestible capsule device 10 and the receiver apparatus 30. The internal electronics may further comprise an accelerometer 19 from which accelerometer data (i.e. a signal) is received at the processor hardware 151 for processing and subsequent storage at the memory hardware 152 and transmission by the wireless transceiver 18.

The gas sensors 131, 132 are less than several mm in dimension each and are sensitive to particular gas constituents including oxygen, hydrogen, carbon dioxide and methane. In fact, the VOC gas sensor 132 may be configured to give sensor side readings and driver or heater side readings. The heater side readings may be used to determine thermal conductivity of a surrounding gas and thereby the heater side readings of the VOC gas sensor are TCD readings. The sensor side readings are used to determine concentrations of volatile organic compounds in the surrounding gases and are VOC readings. The TCD gas sensor 131 may be, for example, a heating element coupled to a thermopile output, with the thermopile temperature, and therefore its output, varying due to energy conducted into the gas at the location of the capsule 10. The TCD gas sensor 131 measures rate of heat diffusion away from the heating element.

As illustrated in FIG. 3, the heater side of the VOC gas sensor 132 (operating as a TCD sensor) and the sensor side of the TCD gas sensor 131 have different operating ranges, so TCD readings from the two sensors collectively span a wider range of operating temperatures than either of the sensors individually. Both sensors have heating elements. The TCD gas sensor 131 may have a lower operating temperature but with a higher precision. The heater side of the VOC gas sensor 132 increases the operating range but has a lower precision for TCD readings than the TCD sensor. The larger collective thermal range achieved by the two gas sensors 13 in concert enables better resolution of analytes in the event that the signals from the gas sensors are processed to resolve the analytes. The thermal conductivity of constituent gases in the gas mixture of the GI tract varies with temperature and so by obtaining TCD readings at different operating temperatures the different gases can be resolved from each other. This is leveraged in a gas resolution processing branch, which is to determine identity and concentrations of constituent gases in the gas mixture surrounding the capsule 10. The gas resolution processing may be performed on-board the gas capsule 10, at the receiver apparatus 30, or at a remote processing apparatus. The gas resolution processing is optional depending on the implementation. For example, raw readings of a TCD gas sensor 131 may themselves provide sufficient information for presence of SIBO to be determined without processing to resolve changing concentrations of specific gases.

The gas sensor hardware 13 is contained in a portion of the capsule 10 sealed from the power source 16 and other electronic components by an internal membrane 111. Such an arrangement minimises volume of the sensing headspace (i.e. the sealed portion) and minimises risk of a leak caused by a perforated membrane allowing GI-tract gases from the headspace to reach the power source. However, since the power source (and other internal electronics) may be configured so that exposure to GI-tract gases does not adversely impact performance, the internal membrane may be omitted. That is, the internal membrane 111 is optional depending on design and specifically selection and configuration of internal electronic components. The internal membrane 111 is permeable by electronic circuitry required to connect the components housed on either side. For example, wiring may pass through the membrane 111 in a sealed manner. The outer surface of the sealed portion of the capsule is composed of or includes a part that is composed of a selectively permeable membrane. Selectively permeable in the present context indicates that liquids are not allowed to permeate whereas gases are. The selectivity may extend to allowing only a subset of gases to permeate. For example, the gas sensor hardware 13 may include a heater or heaters which are driven to heat sensing portions of the gas sensor or respective gas sensors to temperatures at which sensor readings are obtained (i.e. a measurement temperature). The heater or heaters may be driven in pulses so that there is temporal variation in the sensing portion temperature and so that measurement temperatures are obtained for periods sufficient to take readings but without consuming the power that would be required to sustain the measurement temperature continuously.

The gas sensors 13 may be calibrated, so that a gas sensor reading can be used to identify the composition and concentration of a gas to which they are exposed. Calibration coefficients are gathered in manufacturing and testing, and are applied to the recorded readings at the processing stage (i.e. by a server such as on the cloud or by an on-board processor 151). Otherwise, this calibration could be performed on the capsule 10, at the receiver apparatus 30, or on any device having access to the calibration coefficients and the recorded readings from the gas sensors 13. Such calibration relates to a gas resolution branch of processing concerned with measuring the concentration of constituent gases in the gas mixture at the capsule 10. Context for the outputs of that branch of processing is provided by a motility branch of processing, which determines (or predicts to within predefined confidence level) a location of the capsule 10 within the GI tract at which said gas mixture is found. In the motility (or location determination) processing branch, some calibration may also be required in seeking to find gastric-duodenal transition indicators, since ingested foodstuffs at different temperatures change the environmental temperature in the stomach, which influences rate of heat diffusion.

Calibration of the gas sensor hardware readings to resolve particular constituent gas or gases is not required, for example if the metric representing fluctuation or other quantities, and in particular the predefined threshold(s), is set according to the metric representing fluctuation of the raw readings (wherein raw reading is taken to be a value equal to or directly proportional to the signal value obtained from the output of the sensor).

In the case of gas sensor data from readings after ingestion and before the gastric-duodenal transition (i.e. whilst the capsule 10 is in the stomach), processing of readings may include applying a moderation to TCD readings, from either gas sensor, in order to correct for variations in environmental temperature, based on environmental temperature readings by the temperature sensor 14a. TCD readings are effectively measuring rate of heat loss to surroundings, and so accuracy is improved by measuring the temperature of the surroundings rather than by relying on assumption (i.e. prior knowledge of internal temperature of the subject mammal). However, the processing may rely on assumption, for example, if there is some issue with the temperature sensor readings, or, for example, if the level of accuracy provided by assumption is acceptable in a particular implementation. Gastric temperature may vary based on, for example, ingestion of liquids or foodstuffs by the subject mammal, or physical activity undertaken by the subject mammal 40. Environmental temperature is a term used in this document to refer to the temperature of the environment in which the capsule 10 is located, as distinct from operational temperatures of the gas sensors 13. The sensitivity of the gas sensor hardware 13 to different constituent gases may vary according to the operating temperature of the sensors and the processing of the readings includes modifying (also referred to as moderating or correcting) readings from the gas sensors according to contemporaneous operating temperature and optionally also according to contemporaneous environmental temperature.

Processing of the sensor readings may be divided into different branches, for ease of reference and physically insofar as different branches may be performed at different locations, in particular on the capsule 10 or at the receiver apparatus 30 or remote computing device 20. A SIBO determination branch of processing is processing sensor readings to determine presence or absence of SIBO, a motility branch of processing is processing sensor readings to determine timing of motility events (capsule ingestion, capsule gastric-duodenal transition, capsule ileocecal junction transition, and capsule excretion), and a gas resolution branch of processing is to resolve concentration changes of individual gases through the GI tract by combining readings from different sensors (or from a single sensor in different temperature regimes) to resolve individual gases from the mixture. It is noted that the processing branches are not independent of one another. For example, motility indicators (i.e. features or characteristics of sensor output signals used to determine timing of motility events) may be found in readings of concentration of a single analyte gas in the gas mixture at the capsule, obtained by the gas resolution branch of processing and specifically processing the output of one or more of the gas sensors 13. The SIBO determination processing, and in particular the time window from which readings are used in the SIBO determination processing, may be based upon motility events identified in the motility branch of processing.

In addition to the gas sensors 13 and the environmental sensor(s) 14a 14b, the capsule electronics further include processor hardware 151, memory hardware 152, a power source 16, an antenna 17, a wireless transmitter 18, and optionally a reed switch or some other activation mechanism. The wireless transceiver 18 operates in concert with the antenna 17 to transmit readings from the sensors (collectively referring to the gas sensors 13 and the temperature sensor 14a, and optionally also the accelerometer 19 and reflectometer) to a receiver apparatus 30 for processing thereon or at a remote processing apparatus to which the receiver apparatus is in data communication, or the processor hardware 151 processes the readings the sensors to determine presence or absence of SIBO, and/or to identify motility indicators (or otherwise to extract information from the sensor readings), and the result of that processing is transmitted to the receiver apparatus.

The wireless transmitter (also referred to as data transmitter 18) may be provided as part of a wireless transceiver 18. The wireless transceiver 18 includes an antenna 17. Optionally, the wireless transceiver 18 also includes a directional coupler 171. The wireless transceiver 18 may transmit data in accordance with the Bluetooth protocol, the Bluetooth Long Range (Coded-PHY) protocol, the LoRa protocol, the wifi protocol, or using another mode of transmission such as 433 MHz radio wave transmission.

FIG. 2B illustrates the antenna 17 and directional coupler 171 as elements of the wireless transmitter 18, since the antenna is the physical means by which the wireless transmitter 18 transmits data to the receiver apparatus 30. The wireless transmitter 18 is also configured to buffer data for transmission. The wireless transmitter 18 may also be configured to encode the data with a code unique to the capsule 10 among a population of like capsules 10.

Interconnections between electronic components may be via a central bus connection or may be via dedicated connections between pairs of components, or a combination of both. This is one example of how power and data may be distributed between components. A microcontroller may be provided to coordinate distribution of data and power between components. The sensors (from among the TCD sensor 131, the VOC sensor 132, the temperature sensor 14a, the humidity sensor 14b the accelerometer 19, and the directional coupler 171) take readings under the instruction of a microcontroller, powered by the power source 16, and transfer the readings (or results of processing the readings) to the wireless transmitter 18 for transmission to the receiver apparatus 30 via the antenna 17 for off-board processing, or to the processor hardware 151 for on-board processing. For example, the processor hardware 151 and memory hardware 152 may collectively be referred to as a microcontroller.

The dimension of the capsule may be less than 11.2 mm in diameter and less than 27.8 mm in length. The housing of the capsule 10 may be made of indigestible polymer, which is biocompatible. The housing may be smooth and non-sticky to allow its passage in the shortest possible time and to minimise risk of any capsule retention. Optionally, the ingestible capsule may be less than 32.3 mm in length and less than 11.6 mm in diameter.

The antenna 17 may be in series with a directional coupler 171. The directional coupler 171 and the antenna 17 are configured as a reflectometer. The reflectometer measures the amplitude of reflected signals by means of a diode detector. The measurements of the reflectometer are readings that represent electromagnetic properties of material in the vicinity of the capsule. The reflectometer readings provide a basis for differentiating between gaseous, liquid, and solid matter at the location of the capsule in the GI tract. Readings of the reflectometer enable the antenna 17 and directional coupler 171 to operate in cooperation as an environmental dielectric sensor.

The readings of the ingestible capsule 10, which include one or more from among readings from: the temperature sensor 14a, the heater side 132b of the VOC gas sensor 132, the sensor side 132a of the VOC gas sensor 132, and the TCD gas sensor 131, may also include readings of the reflectometer. Hence, change in capsule location within the GI tract causes a change in reflectometer readings, and therefore provide an indicator that a transition event between two sections of the GI tract has occurred.

The ingestible capsule 10 may further comprise an accelerometer 19. The accelerometer 19 may be a tri-axial accelerometer. A rate of change of angular position or orientation of the capsule 10 is somewhat dependent upon location within the GI tract, and therefore accelerometer readings provide an indicator that a transition event between two sections of the GI tract has occurred. The accelerometer readings may measure angular acceleration about three axes of rotation, wherein the three axes of rotation may be mutually orthogonal.

### Processor Hardware, Memory Hardware

The processor hardware and memory hardware may be separate components or may be part of the same single integrated chip. The processor hardware and memory hardware are selected according to the particular implementation requirements of each design or version of the capsule 10, noting that constraints such as power consumption, cost, data throughput, size of data transmission payload, etc, will vary between designs or versions. The processor hardware may be a processor or a plurality of interconnected processors.

### Pairing

The wireless transceiver may be a Bluetooth transceiver, a wifi transceiver, a radio transceiver, or another form of wireless data transceiver. A radio transmitter may be configured to transmit in the 433 MHz band. In any case, the wireless data transmitter may be provided as part of a wireless data transceiver. For example, the wireless data transceiver may receive signals at least in performing pairing or any other form of coupling to a recipient device 30. The capsule 10 may be configured to enter into a wireless pairing or coupling mode immediately upon initiation (i.e. first power-on), wherein a subject or another user is instructed (via written instructions or via an application running on the receiver apparatus 30 itself) to pair or couple the capsule 10 to the receiver apparatus 30 prior to ingestion of the capsule 10. However, the capsule 10 may be configured such that pairing or coupling is not necessary, for example the capsule 10 may be configured to broadcast data to a recipient device in a data transmission technique that is agnostic to pairing or coupling status, as discussed in more detail below.

### Data Transmission Techniques

There are two principal data transmission techniques, which ingestible capsule devices 10 may be configured to use either or both of, depending on implementation details (i.e. use case). In a post-excretion data transmission technique, signals from the sensors are received at the processor hardware 151 (utilising also the storage capabilities of the memory hardware 152) and processed on-board the capsule 10 in order to one or more from among: determine presence or absence of SIBO, identify and record motility indicators (and optionally also other characteristics of the sensor output or sensor readings of interest or groups of sensor readings of interest), and resolve individual gas analytes from the sensed gas compositions, and the processing results (including SIBO determination, recorded motility indicators and optionally also the other characteristics, metrics, and readings or groups of readings of interest, such as peak H2, area under a plot of H2 against time) are stored on the memory hardware 152 as a data transmission payload. Other characteristics and readings or groups of readings of interest may include, for example, maximum or minimum readings from specific sensors or from metrics calculated by combining sensors. For example, metric representing fluctuation which is used to determine presence or absence of SIBO may be stored. Trend line gradient of a single-order polynomial fitted to the gas sensor data may be stored. The maximum or minimum readings may be local maximum or local minimum readings, wherein local is defined by, for example, predefined timings or motility events determined to have occurred by the capsule 10 itself. A specific example is maximum or minimum H2 concentration, which is a metric calculated from the gas sensor readings by an appropriately calibrated processor hardware. The data transmission payload is transmitted by the wireless transceiver once excretion of the capsule 10 from the GI tract is detected (for example by the temperature sensor 14a signal and/or by the accelerometer 19 signal). Metrics further include peak H2 level or value, timing of peak H2, and total H2 (area under the curve). Such metrics may be calculated by the on-board processor hardware 151 during passage through the GI tract of the subject, and transmitted away from the capsule 10 to a receiver device in post-excretion transmission as part of a report or otherwise.

In the post-excretion data transmission technique, the transmission may be via a Bluetooth transmission mode that is not dependent upon pairing status. That is, for example, if the Bluetooth transceiver is paired to a receiver device then it transmits the data transmission payload to the paired receiver device, and if the Bluetooth transceiver is unpaired then it broadcasts the data transmission payload to a recipient device in the absence of pairing in an inquiry mode (which may be referred to as discovery mode or beacon mode). Bluetooth protocol has an inquiry mode in which a device broadcasts a unique identifier, name and other information. The data transmission payload, or part thereof, may comprise or be included in the said other information. In particular, the data transmission payload may be prioritised or otherwise filtered by the processor hardware 151 so that information deemed particular important such as an indication that excretion has occurred (it is important for clinical reasons to know that the capsule 10 has been excreted) and potentially information such as timing of determined motility events, is transferred away from the capsule 10 in preference to other information. Following the inquiry mode transmission, the transceiver may again attempt to pair, connect, or otherwise couple, with the recipient device, and if successful, to transmit the remainder of the data transmission payload. Of course, said pairing, connecting, or coupling, may have been performed initially pre-ingestion so that post-excretion the Bluetooth transceiver is attempting to re-pair, re-connect, or re-couple, with the receiver device 30. It is noted that the present discussion uses Bluetooth as an example of a transmission protocol, but that the same techniques could be applied to different transmission protocols.

In the event that there is data transmission payload pending transmission away from the capsule 10 after the broadcast of the unique identifier, name, and other information during the Bluetooth inquiry mode, then capsule 10 may be configured to initiate or re-initiate a data communication connection (i.e. a pairing or re-pairing) with a receiver device 30. Upon successful initiation or re-initiation of the communication connection, transmission of the said data transmission payload pending transmission away from the capsule 10 is performed whilst the data communication connection remains active.

The Bluetooth transceiver 18, or any other wireless data transmitter 18, may be configured to automatically re-connect following an initial (i.e. pre-ingestion) connection to a receiver device 30. The receiver device 30 may run an app or web app to guide the subject in terms of how to ingest the capsule 10, to notify the subject that the excretion event has been determined, and optionally also that the data transmission payload has been successfully transmitted to the receiver device 30 and so the capsule 10 may be flushed away. It is noted that the terms pair, connect, and couple, are interchangeable in the present document, each representing the establishment of a wireless connection between two devices for wireless data transfer.

It is noted that data transmission payload may be being transmitted throughout passage of the capsule 10 through the GI tract, dependent upon pairing, coupling, or connection to the receiver device 30. However, confirmation that occurrence of an excretion event has been determined by the capsule is information that is of particular importance since safety of capsule 10 is reliant on the capsule 10 being excreted. Therefore, information representing determination of occurrence of the excretion event (i.e. a report thereof) is prioritised and may be transmitted in a broadcast or inquiry mode, whereas the remaining data transmission payload is transmitted once connection between the wireless data transmitter 18 and the receiver device 30 is established. Similarly, in capsules 10 configured to perform SIBO determination processing on-board the capsule 10, the result of the determination of presence or absence of SIBO may be transmitted in a broadcast or inquiry mode.

In Bluetooth inquiry mode, data can be transmitted to the receiver apparatus 30, or to any Bluetooth receiver apparatus within range of the capsule 10, without pairing. The wireless transceiver 18 is operable in a Bluetooth inquiry mode or a Bluetooth long range (Coded-PHY) mode. Capsules 10 may store and transmit among the data transmission payload readings from one or more sensors representing a predefined period such as a period during passage through the small intestine, and optionally also either side of any identified motility indicators. For example, gas sensor signals only, or for all sensors. Such readings may be used for SIBO determination, to add confidence to the identified motility indicators in terms of determining whether or not a motility event has occurred, and/or may provide other information useful in a health or clinical context.

More generally, data transmitted according to the post-excretion data transmission technique may be any of the data transmission payload that has not already been transmitted. For example, the wireless data transmitter 18 may be configured to transmit the data transmission payload to a paired receiver apparatus while still in the GI tract (this transmission is referred to herein as pre-excretion data transmission technique). However, owing to issues such as signal attenuation, noise, power supply issues, temporary pairing failure, or if pairing was never performed in the first place, or for any other reason, some or all of the data transmission payload may be pending transmission at the point of excretion. In that case, the remaining data transmission payload is transmitted according to the post-excretion data transmission technique once excretion is detected. It is noted that down-sampling of the data transmission payload may be performed prior to transmission via the post-excretion data transmission technique. Furthermore it is noted that some elements of the data transmission payload may be prevented from transmission via the post-excretion data transmission technique. For example, since bandwidth, and also time within which to transmit, may be limited, it may be that the motility event indicators and diagnostic indicators themselves are included, but that sensor readings are excluded from the data to be transmitted according to the post-excretion data transmission technique.

In a pre-excretion data transmission technique, the sensor signals are transmitted continuously by the wireless transceiver 18. In the pre-excretion data transmission technique, the process hardware 151 coordinates the receipt of the signals from the sensors and the storage at the memory hardware 152 for transmission by the wireless transceiver 18.

In the example of a Bluetooth wireless transceiver 18, in the pre-excretion transmission technique the transceiver may be operated according to a long-range or coded-PHY Bluetooth transmission procedure, such as BTLE Coded PHY. A signal power enhancement of around 10 dB is achievable via BTLE Coded PHY Bluetooth transmission procedure.

During a data transmission phase of the ingestible capsule 10 (i.e. which in the post-excretion data transmission technique is in a short burst post-excretion and in the pre-excretion data transmission technique is continuous while the ingestible capsule 10 is in use, that is, in the GI tract of a subject mammal 40 and obtaining and transmitting readings) the wireless transmitter 18 transmits the readings to a receiver apparatus 30, which may be a dedicated device for receiving and storing the readings (and optionally with a user interface) or may be a multi-function device such as a mobile phone (such as a smart phone). The mobile phone may be running an application which processes some or all of the data transmission payload to determine presence or absence of SIBO in the patient, and/or to generate a motility report or diagnosis of a medical condition based on motility indicators and diagnostic indicators either included in the data transmission payload or derivable therefrom. Alternatively, the application may be configured to transmit the data transmission payload on to a server 20 or another processing apparatus to determine the presence or absence of SIBO or to generate the motility report or diagnosis based on the data transmission payload. The subject mammal need not remain within a specific range of the remote computer 20 during the live phase. Capsules 10 equipped with a Bluetooth transceiver 18 may communicate directly with a smartphone of a user, which obviates any need for a dedicated receiver apparatus (the smartphone taking on the role of receiver apparatus 30). The receiver apparatus 30 (whether a dedicated device or a mobile phone or tablet computer) may process the readings itself or may upload the readings to a remote computer 20 for processing (i.e. determining presence or absence of SIBO, identifying motility indicators, determining motility event timings, resolving gas analytes). The upload may be continuous during a live phase of the capsule, or the upload may be after the live phase of the capsule is terminated. The receiver apparatus 30 may also store the readings, so that loss of connectivity between the receiver apparatus 30 and a remote processing apparatus is not critical.

The on-board processor 151 may apply one or more processing or pre-processing steps, as discussed in more detail below. Digitisation of the readings is performed either by the sensors themselves, by the processor 151 or by the wireless transceiver 18. The digitised readings are transmitted via the antenna 17. The readings of the capsule 10 are made at an instant in time and are associated with the instant in time at which they are made. For example, a time stamp may be associated with the readings by the microcontroller 15, the wireless transmitter 18, or at the receiver apparatus 30 or remote computer 20. For example, if readings are made and transmitted more-or-less instantaneously (i.e. within one second or a few seconds) by the wireless transmitter 18 then the time of receipt by the receiver apparatus may be associated with the readings as a time stamp. Processing of the readings discussed further below is somewhat dependent on the relative timings of the readings (i.e. so that contemporaneous readings from the different sensors can be identified as contemporaneous), however accuracy to the level of one second, a few seconds, or 10 seconds, is sufficient.

In a hybrid mode, capsules 10 may combine the two data transmission techniques. For example, the capsule 10 may process sensor readings on-board to identify motility markers (and optionally also other readings or groups of readings of interest) for transmission in Bluetooth inquiry mode immediately post-excretion. In addition, the capsule 10 may continuously transmit sensor readings to a paired receiver apparatus. Optionally, the continuous transmission may be of the gas sensor data only, or gas sensor data and environmental sensor data (being one or more from among environmental temperature sensor data and relative humidity sensor data) required to calibrate gas sensor signals or otherwise to assist in motility event detection. Gas sensor data is of particular interest in providing health and clinical information, particularly once combined with motility indicators provided by the other sensors such as accelerometer, reflectometer. Gas sensor data may be downsampled or subject to other compression techniques by the on-board processor prior to transmission. Optionally, the on-board processor hardware 151 may apply one or more filters, such as a high pass or low pass filter applied to the values themselves or to the derivative with respect to time, so that only gas sensor data meeting particular thresholds is included in the data transmission payload. Metrics representing gas sensor data, such as peak of a derived H2 value, or area under a plot of derived H2 value with respect to time, may be maintained and transmitted away from the capsule 10.

For capsules 10 configured to perform data transmission during passage through the GI tract (i.e. pre-excretion data transmission technique), commercial bands (such as 433 MHz) may be used by the antenna 17 as electromagnetic waves in this frequency range can safely penetrate the mammalian tissues 40. Bluetooth may also be used in such capsules, wherein Bluetooth may be long-range Bluetooth, particularly when BMI of the subject (human) is above a threshold, or a high level of attenuation is expected for some other reason. Other commercial bands and protocols may be used in various applications, such as LoRa. Coding may be applied at the digitisation stage to assure that the data transmitted by the capsule 10 is distinguishable from data transmitted by other similar capsules 10. The transmission antenna 17 may be, for example, a pseudo patch type for transmitting data to the outside of the body data acquisition system.

Power source 16 is a battery or super capacitor that can supply the power for the sensors and electronic circuits including the processor hardware 151 and memory hardware 152. A life time of at least 48 hours may be set as a minimum requirement for digestive tract capsules. A number of silver oxide batteries in the power source 16 is configurable, depending on the needed life time and other specifications for the capsule. For example, long-range Bluetooth may consume more power than standard Bluetooth. Capsules 10 may be configured to switch from long-range Bluetooth transmission to standard Bluetooth transmission once the stored energy in the battery (or batteries) drops below a predefined threshold, wherein the on-board processor or microcontroller is configured to monitor stored energy level.

### Data Processing Approaches

The on-board sensors generate a large amount of data. Limitations such as energy capacity of power source mean that it may be preferable to process some data on-board the capsule 10 in order to extract a (relatively smaller) data transmission payload from the (relatively larger) generated data. In addition to extraction, data processing techniques may summarise or otherwise represent the generated data in order to reduce the size of the data transmission payload. The processor hardware 151 may be configured to prioritise contents of the data transmission payload. In particular, data representing that the excretion event has been determined and the timing thereof may be given highest priority (i.e. transmitted in preference to other content of the data transmission payload pending transmission at the same time as the data representing that the excretion event is pending transmission).

It will be appreciated that there is a fill spectrum of possibilities between, at one extreme, transmitting all generated data away from the ingestible capsule device 10 for processing elsewhere (i.e. from capsule perspective a high data transmission burden and low data processing burden) and at the other extreme performing a high degree of processing on board to determine results including: presence or absence of SIBO, timings of motility events to a high degree of certainty, diagnosis of specific health conditions or ailments, and only transmitting the said processing results (i.e. from capsule perspective a low data transmission burden and high data processing burden).

A specific example is processing sensor readings on-board the capsule 10 to determine timing of gastric-duodenal transition and ileocecal junction transition (i.e. so that timing of passage through small intestine is determined), and then to include in the data transmission payload gas sensor data from readings taken in the period between the two determined timings (along with data from environmental temperature sensor if required for compensation), but to exclude from the data transmission payload gas sensor data from periods outside of the said period.

Embodiments are configurable at the design stage according to implementation requirements to combine data processing and data transmission in a manner that enables data processing to occur, whether on-board the capsule 10, at a receiving apparatus 30, or at a remote data processing apparatus 20, to determine any from among presence or absence of SIBO, motility events, and other gut health indicators such as gas constituent concentrations at one or more locations/timings in the GI tract, and to identify or detect diagnostic indicators.

The term signal may refer to the output signal produced by a sensor, whereas the term reading may refer to a specific measurement of the signal taken at or otherwise associated with an instant in time, which instant in time may be included with or associated with the reading explicitly or implicitly (i.e. if the reading is the 1000^{th} reading in a series and readings are taken at a rate of 1 Hz and the timing of the first reading in the series is known, then the position of the reading in the series implicitly represents the timing). The term data when applied to sensors is taken to mean data embodying those readings or signals, noting that the data may be processed, for example to compensate for effects of environmental temperature variation. Time stamps or other timing indicators may be provided by the processor hardware 151. Data represents a reading as a value or a vector comprising plural components, such as one for timing, one for reading value, and optionally further information such as sensor temperature at time of reading, etc.

On-board processing may be performed in more-or-less real time, allowing for latency caused by transfer between components and processing itself. Alternatively, the readings may be received by a receiver apparatus 30 processed thereby and/or stored for upload and processing retrospectively by a remote processing apparatus 20 Dependencies may exist between indicators or markers in the data which constrain an order in which readings are processed.

The on-board processor 151 may be configured to perform methods, such as those illustrated in FIGS. 4A to 4H, by executing processing instructions stored on the on-board memory hardware 152. Data processing overhead is increased in this case, which increases performance requirements and thus cost of the on-board processor 151 and memory 152, but reduces the data transmission overhead thus suppressing performance requirements of the wireless data transmitter 18. On the assumption that processing data on-board consumes less energy than transmitting said data to a receiver apparatus 30 for off-board processing, the on-board processing case also reduces stored energy requirements at the power source 16.

In the off-board processing case, the processing may be executed at a receiver apparatus 30 in direct communication with the ingestible capsule device 10, or at a computing apparatus 20 in data communication with the receiver apparatus 30. For example, the receiver apparatus 30 may be a dedicated device configured to receive signals transmitted by the wireless data transmitter 18, such as signals transmitted in the 433 MHz radio band. Alternatively the receiver apparatus 30 may be a general purpose computing apparatus such as a smartphone or tablet computer configured to receive signals transmitted by the wireless data transmitter 18, such as signals transmitted according to the Bluetooth transmission protocol, or according to the LoRa transmission protocol.

Communication between the capsule 10 and the receiver device 30 may be via a wireless data transmitter 18 on the capsule 10 configured to transmit signals according to the LoRa data transmission protocol.

Communication between the capsule 10 and the receiver device 30 may be via a wireless data transceiver 18 on the capsule 10 configured to transmit signals according to the Bluetooth data transmission protocol.

Communication between the capsule 10 and the receiver device 30 may be via a wireless data transceiver 18 on the capsule 10 configured to transmit signals according to the Bluetooth long-range (coded-PHY) transmission protocol.

Specifically the signals transmitted according to the Bluetooth transmission protocol may be transmitted according to a post-excretion transmission mode, being a term referring to a transmission mode that does not depend upon paired status, by virtue of broadcasting data, or by virtue of initially attempting to transmit data to a couple/paired device but broadcasting data as a fallback in case coupling/pairing is unsuccessful. Broadcasting data may be executed in a handshake mode, inquiry mode, or discovery mode, in which data is broadcast by the data transmitter. For example, a generated report such as illustrated in FIG. 4D at step S50 may be included in broadcast data.

In the post-excretion transmission mode, the wireless data transmitter may initially attempt to pair to a receiver, and implement the broadcasting if the pairing attempt is unsuccessful. The pairing attempt may be an attempt to re-pair to a receiver that has previously been paired to the transmitter. Data may be transmitted according to a coded-PHY Bluetooth transmission protocol, or according to a standard Bluetooth transmission protocol.

In the post-excretion transmission mode example, excretion of the ingestible capsule device 10 from the subject may be detected by an on-board environmental temperature sensor 14, the measurements, signal, or readings of which are monitored by the on-board processor 151 which triggers the beacon transmission mode of the wireless data transmitter 18 to transmit a data transmission payload immediately upon detection of capsule excretion.

The post-excretion transmission mode may be triggered by determination that an excretion event has occurred (i.e. that the capsule has been excreted) based on readings of an on-board temperature sensor, and specifically a decrease from the in-vivo temperature. In a case in which the capsule device 10 has already been paired to a receiver 30 such as a smartphone, for example during an initiation procedure, the capsule device 10 may attempt to re-pair, and if successful, transmit a data transmission payload to the paired receiver 30. In the event of re-pair being unsuccessful, for example after a finite number of attempts or after a timeout (for example, 1 second, 3 seconds, 5 seconds), the wireless data transmitter 18 is configured to transmit a data transmission payload in a discovery, inquiry, or handshake mode, which is ordinarily a pre-cursor to pairing and enables some data transfer. A dedicated application at the receiver 30 is configured to access and process the data transmission payload so transferred.

An excretion event may also be detected by monitoring the readings from a relative humidity sensor for a step increase readings from the relative humidity sensor associated with exit from the rectum and submersion in a toilet bowl.

The data transmission payload may comprise one or more from among: a diagnosis outcome (positive/negative), an indication that SIBO has been determined to be present in the subject, an indication that SIBO has been determined to be absent in the subject, an indication that no determination could be made as to presence or absence of SIBO in the subject, a measured level of fermentation activity measured in the small bowel of the subject, and one or more calculated metrics or parameters leading to the diagnosis, detection, determination, or measured level. In a further example, the data transmission payload may comprise a representation of a predefined characteristic feature in the readings generated by a specific gas sensor such as the TCD gas sensor, whether that representation be the underlying readings from the specific gas sensor, or a parameter derived therefrom such as an indication of presence/absence of an increase in concentration of a particular component in the gas mixture. An example of a predefined characteristic feature is metric representing fluctuation, such as aggregate fluctuation, during passage through the small intestine. A further example is gradient of a first-order polynomial trend line fitted to the gas sensor data from readings taken during passage through the small intestine.

The present method for determining presence of SIBO was developed in specific trials and other data-gathering exercises in which ingestible capsule devices 10 such as disclosed in Australian patent application number 2022900873 and predecessor versions thereof (all housing gas sensor hardware inter alia other sensor devices and electronic components) are ingested by subjects (some having positive SIBO diagnoses based on other tests such as jejunal aspirate test and some having negative diagnoses) and the data generated by the on-board sensors analysed to identify characteristics or features that are indicative of SIBO.

### Description of Methods in FIGS. 4A to 4H

FIGS. 4A to 4H illustrate methods for diagnosing SIBO in a patient based on data generated by sensors on board an ingestible capsule device 10 ingested by the patient. The method of any of FIGS. 4A to 4H may be computer-implemented. The method of any of FIGS. 4A to 4H may be executed by processor hardware 151 in cooperation with memory hardware 152 on board an ingestible capsule device 10. The method of any of FIGS. 4A to 4H may be executed by a receiver device 30 configured to receive data representing a time series of readings from gas sensor hardware housed within an ingestible capsule device 10 from the ingestible capsule deice 10, or by a remote computing apparatus 20 in data communication with such a receiver apparatus 30. The method of any of FIGS. 4A to 4H may be executed by a combination of one or more from among: the processor hardware 151 on board the ingestible capsule device 10, the receiver apparatus 30, and/or the remote computing apparatus 20.

FIGS. 4A to 4H illustrate methods for determining presence of small intestinal bacterial overgrowth, SIBO.

### Step S10: Obtaining Data

At step S10 data is obtained representing a time series of readings from gas sensor hardware housed within an ingestible capsule device 10 orally ingested by a subject 40, the time series of readings being taken during exposure of the gas sensor hardware to a gas mixture at the ingestible capsule device 10 during passage of the ingestible capsule device through a gastrointestinal tract of the subject 40, each reading representing a composition of the gas mixture at the location of the ingestible capsule 10 device in the gastrointestinal tract of the subject 40. In particular, the data represents gas sensor readings taken during passage through the small intestine, though it is noted that the data may represent readings from a longer timeframe with cropping applied retrospectively once timing of passage through the small intestine is determined. Obtaining data S10 may be by receiving the data from the sensor hardware itself or from, for example, a sampler configured to periodically sample an output signal from sensor hardware. Obtaining data S10 may be by receiving data from the ingestible capsule device 10 itself. Obtaining data S10 may be by reading data from a predefined storage location. The time series of readings may be time-stamped values, or the temporal component may be implicit via a placement in a chronological sequence of readings. The readings may be taken at predefined intervals, such as every second, every 5 seconds, every 10 seconds, every 15 seconds, every 20 seconds, every 30 seconds, every minute. The readings form a time series. The readings may each include an explicit indication of time such as a time stamp, or time may be implicit by virtue of position within a chronological sequence. For example, post-initiation, the nth reading is at a time of n×m seconds, wherein m is the period between successive readings.

The gas sensor hardware may include, for example, an H2 gas sensor specifically sensitive to changes in concentration of H2 in the gas mixture at the location of the ingestible capsule device 10. The gas sensor hardware may include, for example, a CH4 gas sensor specifically sensitive to changes in concentration of CH4 in the gas mixture at the location of the ingestible capsule device 10. The gas sensor hardware may include, for example, a CO2 gas sensor specifically sensitive to changes in concentration of CO2 in the gas mixture at the location of the ingestible capsule device 10.

The gas sensor hardware may comprise, for example, a TCD gas sensor 131 sensitive to changes in thermal conductivity of the gas mixture at the location of the ingestible capsule device 10, which is correlated with changes in concentration of different constituent gases. The TCD gas sensor 131 may be operated at a single operating temperature or at multiple operating temperatures, and thus by taking TCD gas sensor readings at different operating temperatures and based on the correlation, concentrations of different composite gases are derivable. The gas sensor hardware may be or may include, for example, a VOC gas sensor 132 sensitive to changes in concentration of volatile organic compounds in the gas mixture at the location of the ingestible capsule device 10.

The processor executing the method may be on-board the ingestible capsule device 10, or off-board, wherein off-board includes being either at a receiver apparatus 30 in direct communication with the ingestible capsule device 10, or at a remote apparatus 20 in data communication with the receiver apparatus 30.

Optionally, the ingestible capsule device further comprises processor hardware 151, memory hardware 152, and a wireless transmitter 18, and the processor hardware 151 in cooperation with the memory hardware 152 is configured to perform the method of FIGS. 4A to 4H during passage of the ingestible capsule device 10 through the gastrointestinal tract of the subject 40, and further to diagnosing SIBO in the subject, to transmit data indicating the diagnosis to a receiver device via the wireless transmitter. The processor hardware and memory hardware may be combined in a single chip.

### Step S20: Calculate Metric Representing Fluctuation

At S20, gas sensor data is used to calculate metric representing fluctuation, such as aggregate fluctuation, of concentration of a gas or gases. Fluctuation is difference between a value of a time series data point and a contemporaneous value of a trend line fitted to the time series data, the trend line being for example a first-order polynomial. Aggregate fluctuation is summation of the magnitudes of said differences over all relevant time series data points (i.e. all belonging to the pertinent time period). Calculating a metric representing fluctuation is discussed in more detail below with reference to data from live trials.

The gas sensor data may be, or be in direct proportion to, sampled values of an output signal generated by the gas sensor hardware.

The gas sensor data may be obtained by processing sampled values of an output signal generated by the gas sensor hardware to extract a contribution from a specific gas, the gas sensor data being the extracted contribution from the specific gas.

The gas sensor data may be obtained by obtaining readings from an environmental temperature sensor housed within the ingestible capsule device representing environmental temperature at the ingestible capsule device, and compensating sampled values of an output signal generated by the gas sensor hardware to account for variations in environmental temperature, the gas sensor data being the compensated values.

The metric representing fluctuation, such as aggregate fluctuation, at S20 may be calculated from gas sensor data representing readings from an individual gas sensor. For example, a TCD gas sensor 131. Alternatively, the metric representing fluctuation, such as aggregate fluctuation, may be calculated from gas sensor data representing readings from plural gas sensors including one or more from among: a TCD gas sensor, plural TCD gas sensors having different sensitivity levels, plural TCD gas sensors having different sensitivity levels at different operating temperatures, a VOC gas sensor, a dedicated H2 gas sensor, a dedicated CH4 gas sensor.

### FIG. 4A: S40 Determine Presence of SIBO in the Subject

At S40 the metric representing fluctuation, such as aggregate fluctuation, is used to determine presence or absence of SIBO in the subject. For example, the metric representing fluctuation may be compared with a predefined threshold, such as illustrated at S30 in FIGS. 4B to 4D.

Alternatively, SIBO may be determined by combining the metric representing fluctuation with past values of the same metric calculated for the same patient using the same method (noting that capsules 10 are single-use so plural capsules would be required). Said combination may be a straightforward summation or average with the current result and one or more past results. Alternatively a weighted average may be calculated by including a time-decaying weighting according to age (so that more recent results carry a relatively higher weight than less recent results).

Optional step S50 of the method of FIG. 4A is generating and outputting a report including the determination of presence or absence of SIBO from step S40. Outputting may be transmitting to the receiver apparatus 30 by the ingestible capsule 10, or presenting on a GUI on a display unit of the receiver apparatus 30 or remote computing apparatus 20. Outputting may be by the receiver apparatus 30 or remote computing apparatus 20 and include generating and transmitting to a clinician and/or the patient a message such as an email, SMS, or some other message format to communicate the result of the determination at S40.

Step S30: Compare Metric Representing Fluctuation with Threshold

In FIGS. 4B to 4D, at S30 the method includes a first comparison to determine whether or not the metric representing fluctuation calculated or measured at S20 meets a predefined threshold. The predefined threshold is calculated in testing using data obtained from live testing in capsules given to patients known (based on best available testing processes) to either have SIBO or not, and establishing a threshold that is determinative of presence of SIBO if exceeded, and optionally also a threshold that is determinative of absence of SIBO if not exceeded (noting that the two thresholds may be equal).

### Determining Relevant Lime Period—Timing of Passage Through Small Intestine

The methods detect a fermentation indicator or some characteristic, parameter or value among readings taken during passage of the ingestible capsule device 10 through the small intestine. Timing of passage through the small intestine may be determined by positively determining that the capsule 10 is in the small intestine (i.e. sensor readings having values or displaying characteristics consistent with presence in the small intestine), or by detecting timing of gastric-duodenal transition and detecting timing of an ileocecal junction transition indicator (i.e. entry into and exit from the small intestine is detected so that readings between those two events can be attributed to presence in the small intestine). The timing of the ileocecal junction transition indicator provides an upper bound of the timing of readings which are processed to identify a fermentation indicator, and the lower bound may be set by a fixed duration preceding the ileocecal junction transition indicator, or the lower bound may be determined by detecting gastric emptying, i.e. gastric-duodenal transition of the ingestible capsule device 10 into the small intestine. The timing of the gastric-duodenal transition may be the lower bound. Optionally, buffers or cushions may be applied, for example so that the relevant period starts a fixed period after detected gastric-duodenal transition timing and ends a fixed period before detected ileocecal junction transition timing. An ileocecal junction transition indicator detected in the gas sensor data may be referred to as a gas sensor data ileocecal junction transition indicator.

Gastric-duodenal transition may be detected by processing TCD gas sensor readings, for example. A gastric-duodenal transition indicator detected in the gas sensor data may be referred to as a gas sensor data gastric duodenal transition indicator. More detail is provided below on detecting the gastric-duodenal transition indicator. Alternatively, readings from a sensor such as an accelerometer 19 or a reflectometer 18, or both in combination, may be utilised to detect presence of the ingestible capsule device in the small intestine and thus to determine the period from which readings are processed in step S20. For example agitation of the ingestible capsule increases in the small intestine relative to the stomach and this is represented in the output signal of the accelerometer 19. Similarly the dielectric constant of the stomach is different to that of the small intestine and hence readings from a reflectometer 18 can be processed to detect that the capsule 10 is in the small intestine. A gastric-duodenal transition indicator detected in the accelerometer data may be referred to as an accelerometer data gastric duodenal transition indicator. A gastric-duodenal transition indicator detected in the reflectometer data may be referred to as a reflectometer data gastric duodenal transition indicator.

Exit from the small intestine (ileocecal junction transition) may be detected by, for example, changes in the accelerometer and/or reflectometer readings. More than one detected indicator may be combined to determine timing of ileocecal junction indicator. An ileocecal junction transition indicator detected in the accelerometer data may be referred to as an accelerometer data ileocecal junction transition indicator. An ileocecal junction transition indicator detected in the accelerometer data may be referred to as an accelerometer data ileocecal junction transition indicator.

### FIGS. 4B to 4D: S40 Determine Presence of SIBO in the Subject

In FIGS. 4B to 4D, at step S40, the method includes, in response to the first comparison, determining presence, or absence, of SIBO in the subject. For example, the first comparison alone may be sufficient to determine that SIBO is present in the subject. Or, it may be that a further threshold is to be satisfied, such as a threshold applied to a trend line gradient, as illustrated in FIG. 4C. In any configuration, preprocessing in trials and/or using published data is performed to determine a threshold value deterministic of SIBO in the metric representing fluctuation and optionally other SIBO indicator such as trend line gradient. FIG. 4C may be referred to as a two-threshold method.

FIG. 4C illustrates a method in which two criteria are applied to determine presence of SIBO in a patient: firstly at S30 whether or not the metric representing fluctuation exceeds a predefined threshold; and secondly at S32 whether or not a trend line fitted to the gas sensor data at S22 has a gradient exceeding a further predefined threshold. For example, both thresholds being exceeded is determinative of presence of SIBO. Optionally, one but not the other being met may be indicative of SIBO being possibly present but further testing is required. Both not being met may be determinative of absence of SIBO. The predefined thresholds are set to diagnose to a confidence level such as 90%, 95%, or 99%, based on trials and published data. The line from S22 to S20 illustrates that the trend line from S22 may be used as the reference line for calculating metric representing fluctuation, such as aggregate fluctuation, at S20.

### Step S22: Calculate Trend Line Gradient

At step S22 (for example FIGS. 4D, 4E, 4G) a best fit line is fitted to the gas sensor data from the relevant period. The relevant period being at least a portion of the time during which the capsule 10 is present in the small intestine of the subject. Optionally, outliers may be removed before the best fit line is determined. The best fit line may be constrained to being a first order polynomial. There is no constraint on y-axis intercept. The best fit line may be referred to as a trend line. The best fit line may be determined by using a least squares method.

### Step S32: Trend Line Gradient Vs Threshold

The trend line gradient threshold may be a gradient on the magnitude of the gradient, so that it does not matter whether the gradient is positive or negative. FIG. 11B is an example of data from a patient showing as positive for SIBO via jejunal aspirate testing and with a trend line that is negative, but steep enough to exceed a threshold. Alternatively, the threshold for the trend line gradient may be a negative value, wherein trend line having a gradient more negative than the threshold is considered to meet or exceed the threshold. Alternatively, the threshold for the trend line gradient may be a positive value, wherein trend line having a gradient more positive than the threshold is considered to meet or exceed the threshold.

### FIG. 4E: S40 Determine Presence of SIBO in the Subject

FIG. 4E illustrates a method which at S40 combines two characteristics of the gas sensor data to determine presence or absence of SIBO in the subject. Specifically, FIG. 4E at S40 calculates a weighted average or weighted sum combining metric representing fluctuation from S20 and trend line gradient from S22 with respective weightings. The weightings and a threshold value of the weighted sum or weighted average determinative of presence of SIBO are determined using data from live trials and clinical standard aspirate results giving true status of trial subjects in terms of SIBO positive or negative. The weighted sum or weighted average may also be referred to as a weighted multi-factorial metric.

A weighted average or weighted sum is an example of how the sensor data from the capsule 10 may be processed to determine presence or absence of SIBO. The weighted average is based on sensor readings generated by sensors or pseudo-sensors (reflectometer) on board the capsule 10 during passage through the small intestine. The weighted average may be calculated by combining two factors with respective weights applied: the metric representing fluctuation, such as aggregate fluctuation, and the trend line. The weighted average may be calculated only from those two factors and their respective weights. The weighted average may take into account additional factors, which additional factors are characteristics of data from sensors or pseudo-sensors on board the capsule 10. The respective weights may be predefined based on data obtained in trials with known clinical-standard SIBO diagnoses and configuring the weightings and a threshold weighted average to distinguish SIBO positive patients from others. The weightings themselves may be entirely preconfigured, or may be preconfigured to a range, with a value within that range being selected adaptively according to a characteristic of the sensor data such as noise. It is noted that weighted sum and weighted average are interchangeable in the present context.

### Step S50: Outputting Report of Determination

FIGS. 4A, 4D,4E, 4G, 4H illustrate that the methods may comprise one or more further steps: generating a report of the determination of presence or absence of SIBO; and outputting the report. Outputting the report may comprise displaying or printing the report in the case that it is generated by a receiver apparatus 30 or remote computing apparatus 20 either having, or connected to, a display device and/or a printing device. In the case that the report is generated on board the capsule 10 itself, the outputting is by transmitting the report away from the capsule 10 to a receiver apparatus 30 and optionally on to a remote computing apparatus 20. Noting that the report generated at S50 may comprise only the result of the determination, may include further information such as the metric representing fluctuation calculated at S20 and/or the trend line gradient from S22, and may include further information such as timing of passage of the capsule 10 through the small intestine.

Further data may be included in the report such as one or more from among: readings forming the ileocecal junction transition indicator, or a representation thereof, readings forming the gastric-duodenal junction indicator, or a representation thereof, and readings forming an excretion indicator, or determined timing of excretion, or data representing that excretion of the capsule 10 by the subject has been positively determined.

Use of dashed lines for steps S22 and S32 in FIG. 4D illustrates that those steps are optional: if included the method of FIG. 4D is the method of FIG. 4C with the additional reporting step S50; if excluded the method of FIG. 4D is the method of FIG. 4B with the additional reporting step S50.

The generated report is output, which output may take one or more of a number of different forms. For example, in methods in which the report is generated at the ingestible capsule device 10 the generated report is output to a receiver device 30 via the wireless data transmitter, either during passage through the remainder of the GI tract of the subject, or upon detection of excretion. In methods in which the report is generated by the receiver apparatus 30 or remote processing apparatus 20 in data communication therewith, the output may be transmission to a clinician and/or patient via a messaging interface, or the output may be display of the report on a user interface.

The level of fermentation activity in the small bowel may be included in the report and may be indicated by the metric representing fluctuation. For example, the level of fermentation activity in the small bowel may be measured or calculated by area between a plot of gas sensor data against time and a linear trend line, or, for example are between said plot and the X-axis. Noting, for example, that the gas sensor data may be values of the readings from a TCD gas sensor (corrected to account for environmental variation), or the gas sensor data may be values derived from those readings such as H2 concentration. Further, gas concentration data such as H2 concentration or CO2 concentration may be directly measured by a dedicated gas sensor or may be calculated as a derived metric from readings from sensors sensitive to multiple gases such as a TCD gas sensor 131. Level of fermentation activity is a quantification of fermentation occurring in the time series of readings from the gas sensor hardware determined to be taken during residence of the capsule 10 in the small intestine.

Level of fermentation activity is a measurable physical effect of SIBO, noting that factors such as diet, among others, may influence level of fermentation activity. A patient may be monitored over a period of weeks or months to assess effectiveness of a SIBO treatment by performing the method of any of FIGS. 4A-H on distinct occasions and monitoring how the reported level of fermentation activity changes.

Generated reports may include further information such as an indication of location within the small intestine at which fermentation is detected, determined ingestion timing, determined excretion timing, other metrics such as peak hydrogen, total hydrogen, etc.

### Method of FIG. 4F

FIG. 4F illustrates a further method for determining presence or absence of SIBO in a subject.

Step S10 is as discussed above with reference to FIGS. 4A to 4E.

At S102 an ileocecal junction transition indicator is detected among the obtained data, and based on a timing of the ileocecal junction transition indicator, at S103 a fermentation indicator is detected among the obtained data representing readings preceding the timing of the detected ileocecal junction transition indicator. More detail is provided below regarding techniques for detecting the ileocecal junction transition indicator. In addition or as an alternative to being detected in readings from gas sensor hardware, and/or the ileocecal junction transition indicator may be detected in reflectometer data as a reflectometer data ileocecal junction transition indicator.

Detecting the fermentation indicator at S103 may comprise one or both of using gas sensor data to calculate metric representing fluctuation of a concentration of a gas or gases during passage through the small intestine, as discussed above with reference to step S20, and fitting a trend line to the gas sensor data from passage through the small intestine, as discussed above with reference to step S30.

At S104 a determination is made as to whether or not the fermentation indicator is a diagnostic indicator of SIBO. The determination may comprise determining whether either or both of the metric representing fluctuation and the trend line gradient exceed respective thresholds, as discussed above with reference to steps S30 and S32.

At S104 the comparison with the threshold(s) enables a positive or negative SIBO diagnosis to be made, i.e. to determine whether the detected fermentation indicator is a diagnostic indicator of SIBO or not.

### FIG. 4G: S40 Determine Presence of SIBO in the Subject

In the method of FIG. 4G the presence or absence of SIBO is determined from the trend line gradient. FIG. 4G differs from FIGS. 4A to 4E insofar as it does not include a step of calculating metric representing fluctuation, noting that such a step is optional in the context of FIG. 4G.

Steps S10 and S22 of FIG. 4G are as discussed above.

At S40 the best fit line gradient is used to determine presence or absence of SIBO in the subject. For example, the best fit line gradient may be compared with a predefined threshold, such as illustrated at S32 in FIGS. 4C and 4D.

Alternatively, SIBO may be determined by combining the best fit line gradient with past best fit line gradients calculated for the same patient using the same method (noting that capsules 10 are single-use so plural capsules would be required). Said combination may be a straightforward summation or average with the current result and one or more past results. Alternatively a weighted average may be calculated by including a time-decaying weighting according to age (so that more recent results carry a relatively higher weight than less recent results).

Optional step S50 of the method of FIG. 4G is generating and outputting a report including the determination of presence or absence of SIBO from step S40. Outputting may be transmitting to the receiver apparatus 30 by the ingestible capsule 10, or presenting on a GUI on a display unit of the receiver apparatus 30 or remote computing apparatus 20. Outputting may be by the receiver apparatus 30 or remote computing apparatus 20 and include generating and transmitting to a clinician and/or the patient a message such as an email, SMS, or some other message format to communicate the result of the determination at S40.

### FIG. 4H

In the method of FIG. 4H presence or absence is determined based on whether concentration of a specific gas or gases exceeds a predefined threshold concentration for that specific gas.

In FIG. 4H, the gas sensor data obtained at S10 is a specific form of gas sensor data. In particular, the gas sensor data represents concentration of a specific gas or specific combination of gases. Specific in this context signifies known, so that, for example, the specific gas might be hydrogen H2, carbon dioxide CO2, or methane CH4 (or a combination of two of those gases). This is contrasted with cases in which the gas sensor data represents a physical response of gas sensor hardware to the gas mixture which physical response is representative of changes in the composition of the gas mixture as a whole, but which may not be resolved to the specificity of a single gas or combination of gases. The gas sensor hardware may comprise a gas sensor configured to sense a specific gas or gases only and to exhibit nil response to other gases that may be present in the small intestine. Or, the gas sensor hardware may comprise a gas sensor that is sensitive to more gases than the specific gas or gases, and some processing is applied to extract values representing only the concentration of the specific gas or gases for which the predefined threshold to be applied at S24 is configured.

At S24 a predefined threshold is applied to the gas sensor data obtained at S10 and specifically representing concentration of the specific gas or gases at the capsule location during passage through the small intestine. The method of FIG. 4H may be combined with the processing and data transmission techniques disclosed elsewhere in the present specification, and in particular those relating to identifying timing of passage through the small intestine from among the wider set of readings generated by the ingestible capsule device 10.

In particular, the specific gas or gases is a gas or gases that are produced by fermentation in the small intestine, such as one or more of carbon dioxide CO2, hydrogen H2, and methane CH4. Optionally, a minimum number of separate sites or locations of the specific gas or gases exceeding the predefined threshold concentration for the specific gas or gases may be required for a determination of presence of SIBO, such as two or three. Or, for example, the measured concentration of the specific gas or gases is to exceed the predefined threshold concentration for more than a minimum number of readings or for more than a minimum duration for it to be determined at S40 that SIBO is present in the subject. If the criteria for determination of presence of SIBO is not met at S24, then absence of SIBO is determined at S41. If the criteria is met, then presence of SIBO is determined at S40. Optionally, the concentration threshold criteria applied at S24 is not the only determinative factor for presence or absence of SIBO, and the outcome of S24 may be combined with the outcome of one or more other processing techniques, such as comparison of the gradient of the trend line (for example FIG. 4D S22, S32) with a gradient threshold, and the comparison of the metric representing fluctuation with a fluctuation metric threshold (for example, S20, S30, FIGS. 4B, 4C, 4D).

Optionally, a report is generated and output at S50. For example, the report contains at least the result of the determination of the presence or absence of SIBO. The report may further include underlying data for the determination, such as the concentration measurements of the specific gas or gases that exceeded the threshold. The report may further include the timing of those measurements. The report may further include, based on the timing of those measurements relative to the overall timing of the passage through the small intestine, estimate of location in the small intestine of the site or sites of fermentation activity. For example, such estimate may be based on an assumption of uniform displacement per unit time of the ingestible capsule device 10 through the small intestine.

The threshold or thresholds are determined through experiment. The predefined threshold concentration is a fraction or proportion of the specific gas or gases in the overall gas mixture, and as such is independent of gas sensing mode, but does rely upon gas sensor hardware being properly calibrated, and/or accurate determination of gas sensor data representing absolute values of concentration of the specific gas or gases. This is contrasted with other techniques such as the metric representing fluctuation of gas sensor data, which may be based upon gas sensor data representing absolute values of concentration of the specific gas or gases, but alternatively may be based upon readings from a gas sensor sensitive to changes to in composition of the gas mixture but not necessarily directly representing concentration of a specific gas or gases (which may be referred to as raw sensor data).

### Machine Learning

A machine learning algorithm may be trained to perform a method of whether or not gas sensor data from the small intestine indicates presence of SIBO.

Any combination of steps S102 to S104, or S10 to S40, may be performed by an appropriately trained AI classification algorithm. The underlying algorithm may be a convolutional neural network. Training data in the form of gas sensor data from SIBO positive and SIBO negative cases in the PA trial or other supplementary trials with ground truth being a positive or negative diagnosis. The convolutional neural network learns to identify the visual distinction between the gas sensor data in the positive and negative cases and thus to predict whether test cases are SIBO positive or SIBO negative. Using a comparable approach, the neural network may be trained simply to be provided with input vectors comprising two factors: metric representing fluctuation values and trend line gradient value; and to train a classification algorithm to use those two-factor input vectors to classify between SIBO positive and SIBO negative cases.

### Detecting Ileocecal Junction Transition Indicator

Methods may comprise detecting an ileocecal junction transition indicator (referring to transition across the ileocecal junction by the ingestible capsule device 10), which may be detected according to a number of techniques. For example, the ileocecal junction transiting timing may be used to determine an end time of the passage of the capsule 10 through the small intestine, and may also be information that is useful to a clinician in assessing health of the patient GI tract.

Readings of H2 levels may be used as a basis for detecting an ileocecal junction transition indicator at S20. H2 levels may be detected directly by an H2 gas sensor sensitive specifically to changes in H2 concentration. Alternatively, an ileocecal junction transition indicator may be detected by identifying an increase in concentration of volatile organic compounds indicated by VOC gas sensor output exceeding a predefined threshold (either the increase exceeding a predefined threshold or the level itself exceeding a predefined threshold) with a contemporaneous (or temporally adjacent to within a predefined temporal distance either side) increase in H2 levels exceeding a predefined threshold (either the increase exceeding a predefined threshold or the level itself exceeding a predefined threshold). Noting that H2 levels are determined from the TCD gas sensor output and/or heater-side VOC sensor output. H2 levels are determined from TCD gas sensor output by taking TCD readings at different operating temperature setpoints of the TCD gas sensor along with predefined calibration data correlating changes of thermal conductivity at different operating temperature setpoints with variations in concentration of different constituent gases.

Similarly, readings of CH4 concentration may be used as a basis for an ileocecal junction transition indicator. CH4 concentration may be detected directly by a CH4 gas sensor sensitive specifically to changes in CH4 concentration. Alternatively, an ileocecal junction transition indicator may be detected by identifying an increase in concentration of volatile organic compounds indicated by VOC gas sensor output exceeding a predefined threshold (either the increase exceeding a predefined threshold or the level itself exceeding a predefined threshold) with a contemporaneous (or temporally adjacent to within a predefined temporal distance either side) increase in CH4 levels exceeding a predefined threshold (either the increase exceeding a predefined threshold or the level itself exceeding a predefined threshold). Noting that CH4 levels may be determined from the TCD gas sensor output and/or heater-side VOC sensor output.

An ileocecal junction transition indicator may be detected as an increase in concentration of VOCs in the gas mixture at the capsule 10 indicated by the time series of readings from a VOC gas sensor 132. Since an increase in concentration of VOCs in the gas mixture at the capsule 10 may be caused by fermentation in the small bowel, it may be necessary to distinguish one increase from another. Such distinction is by identifying a gradient change, wherein readings proceeding a gradient change are a detected ileocecal junction transition indicator.

FIG. 5 illustrates one technique for detecting ileocecal junction transition: in particular, FIG. 5 illustrates a change in VOC gas sensor readings with a gradient and magnitude, wherein via trials a minimum threshold is established for gradient and magnitude of the change in VOC gas sensor readings associated with transition across the ileocecal junction.

### Detecting Gastric Duodenal Transition Timing

Gastric emptying, gastric-duodenal transition, or crossing the interface between the stomach and the duodenum, may be detected to set a lower bound on timing of passage of the capsule 10 through the small intestine. Such a process is optional since the lower bound may be set by a predefined fixed duration relative to detected ileocecal junction transition timing, or otherwise by detecting presence of the capsule 10 in the small intestine (i.e. not necessarily detecting the transition into the small intestine itself). Gastric duodenal indicator or indicators may be detected in a first subset of recorded readings, the first subset being defined temporally by starting after an ingestion event. Ingestion event may be determined by readings from a temperature sensor 14a (and/or relative humidity sensor 14b) or by a user interaction with an interface on a receiver apparatus 30 or remote computer 20. Furthermore, the first subset may be constrained by sensor, comprising readings from the TCD gas sensor 131. The first subset may further comprise readings from the reflectometer (i.e. the antenna 17 and directional coupler 171) and/or the accelerometer 19.

The gastric-duodenal transition indicator in the TCD gas sensor readings may be a, spike, step change or an inflection point in the TCD gas sensor readings. A correction may be applied to the TCD gas sensor readings to account for changes in environmental temperature, based on recorded readings from the environmental temperature sensor 14a. The gas sensor data may be the values of the output signal from the TCD gas sensor, or may be a temperature-corrected version thereof.

The primary physical mechanism being sensed in the TCD gas sensor readings in detecting the gastric-duodenal transition indicator is as follows: Hydrochloric acid in the gastric juices leaving the stomach mixes with bicarbonate within the bile acids that is released by the pancreas. This bile acid works to neutralize the pH of the liquid and a by-product of this reaction is CO2. In this area of the GI tract the surrounding gases are primarily N2 and O2 with some trace amounts of CO2. The amount of CO2 created in this reaction are significantly higher than the trace amounts that are around due to swallowing of exhaled breath. Therefore, simply using the TCD sensor output without calculating CO2 is appropriate. In other words, the TCD gas sensor readings, once corrected for environmental temperature variations, themselves provide the gastric-duodenal transition indicator, owing to a change in heat conductivity caused by variation in CO2 concentration across the two sides of the gastric-duodenal transition. For motility purposes (i.e. for determining the location of the ingestible capsule 10) there is no particular need to calculate the actual CO2 concentration.

It is noted that the gas sensor data used to calculate metric representing fluctuation, such as aggregate fluctuation, at S20, and the gas sensor data used to detect a gastric-duodenal transition indicator, may be the same or may be different.

As the TCD sensor 131 is affected by the temperature of the gas mixture at the location of the capsule, a temperature correction process is required to account for changes in the external environmental temperature changes i.e. drinking cold water, exercise, eating etc. Starting from the determined ingestion event timing, a bump, step change or large inflection in the readings of the TCD gas sensor 131 plotted against time, not associated with an environmental temperature change, may be a gastric-duodenal transition indicator.

FIG. 6 illustrates recorded readings of an environmental temperature sensor 14a (top line of readings on the top graph) against time, and corrected TCD gas sensor readings against time for an instance of capsule ingestion and progression through a GI tract. The gastric-duodenal transition indicator, which may be labelled gastric emptying, is indicated by a spike above a threshold height in the corrected TCD gas sensor readings. Spike height may be measured, for example, by distance (e.g. as a proportion, as an absolute value, or as a number of standard deviations) from a trend line fitted against the readings up to that point, or from an average value up to that point (wherein the processor maintains an average value). The VOC sensor side trace is marked as Motility (Hot) and illustrates a dip at around 5 hours, which dip is an ileocecal junction indicator.

FIG. 6 shows a gastric-duodenal transition indicator as visible in gas sensor data (TCD gas sensor readings corrected for environmental temperature variation). Thermal conductivity of the gas mixture in the GI tract changes as relative concentrations of gases such as CO2 and H2 vary. CO2 is produced when the hydrochloric acid in the gastric juices leave the stomach and mix with bicarbonate in the bile acids released by the pancreas. This reaction also neutralizes the pH of the liquid. Methods may use the temperature compensated TCD gas sensor data to detect this event, rather than the resolved CO2 contribution, since the TCD gas sensor data contain less noise. The TCD gas sensor raw readings are is adjusted to compensate for the temperature fluctuations measured by the environmental temperature sensor 14a.

As illustrated in FIG. 2B, the circuitry includes a directional coupler 171 in series with the antenna 17, which operate as a reflectometer. A diode detector measures the amplitude of reflected signals from the antenna. The measurements of the diode detector are the reflectometer readings, and measure the reflected energy from the antenna, i.e. energy that was not radiated from the antenna 17 due to impedance mismatches. The reflectometer readings measure the antenna's radiation efficiency which is affected by the dielectric of the material surrounding the capsule.

The readings may become noisy and/or a baseline shift occurs at the timing of the gastric-duodenal transition event. For example, the increase in noise and/or the baseline shift are detectable as transition indicators.

Optionally, an absolute value range may be established for reflectometer readings, wherein readings within the value range indicate presence of the capsule 10 in the small intestine, noting that due to noise, a series of readings may be averaged (optionally after outlier removal) and compared with the value range on a rolling basis to obtain an indication of presence of the capsule 10 in the small intestine.

FIG. 7 illustrates (on the uppermost plot on the lower of the two sets of axes) reflectometer readings against time (labelled “Ant” for antenna), and is marked with the gastric emptying event (GET). The antenna 17 and directional coupler 171 function as a reflectometer to measure the reflected energy from the antenna, i.e. energy that was not radiated out of the antenna. This signal varies as the surrounding dielectric properties change, most notably when the capsule leaves the cavernous fluid filled stomach and transitions to being surrounded by tubular tissue in the small intestine. A shift in the reflectometer readings is observed to be coincident with the GET indicator in the corrected TCD readings (localised spike), adding confidence, as a secondary measure.

FIG. 8 is a plot of recorded readings (or processed versions thereof) against time for a number of sensors and pseudo sensors (reflectometer) in the capsule 10. A gastric emptying (gastric-duodenal transition) event is labelled. The top plot in the graph of FIG. 8 is reflectometer readings against time (labelled “Ant” for antenna). It can be seen that a baseline shift occurs at a time coincident with the spike in corrected TCD gas sensor readings (the spike being a gastric duodenal transition indicator). For example, a baseline shift may be detected by, on a progressive/rolling basis, comparing a mean value of a latest number (e.g. five, ten, or twenty) of consecutive readings, with a mean value of a number of readings preceding (or proceeding in the case of reverse chronological processing) the latest number of consecutive readings. A baseline shift may be indicated by a difference more than a threshold, wherein the threshold may be an absolute value, a proportion, or determined relative to a standard deviation in the readings. Detecting a coincidental gastric-duodenal indicator in the output of the reflectometer may be sufficient to confirm that the first gastric duodenal transition indicator is caused by gastric-duodenal transition of the capsule 10 and thus to determine the timing of the gastric-duodenal transition. Alternatively, the combination of the two indicators may be assessed via a probability model to revise the confidence score and compare the revised confidence score with a threshold, wherein meeting the threshold is to determine that the first gastric duodenal transition indicator is caused by gastric-duodenal transition of the capsule 10 and thus to determine the timing of the gastric-duodenal transition.

As illustrated in FIG. 2B, the capsule 10 may include an accelerometer 19. The accelerometer 19 may provide one or more of a gastric-duodenal transition indicator and an ileocecal junction transition indicator. The indicator provided by the accelerometer 19 may standalone to determine event timing, or may be combined with an indicator from another source to add confidence. An exemplary accelerometer 19 measures roll about three mutually orthogonal axes. The readings from the accelerometer 19 may be vectors with a component per axis, with each component indicating an instantaneous angular acceleration about the corresponding axis, or an average acceleration about the corresponding axis over the time period since the preceding live reading. Alternatively, the readings may give a three dimensional orientation of the capsule. On-board the capsule, at a receiver apparatus 30 or at a remote computer 20, processing of the readings from the accelerometer may be performed to generate a representation (such as a plot vs time) of aggregated (i.e. all three axes) accelerometer readings from which a marker (i.e. a gastro-duodenal transition indicator) is identifiable. Such a plot or representation may also be used to identify markers for other events including excretion event. The processor hardware 151 of the capsule 10, or the receiver apparatus 30 or other remote computing apparatus processing the sensor data, may be configured to process accelerometer sensor data to obtain a time series of a metric (a representative metric) representing the accelerometer data, from which time series one or more from among: a gastric duodenal transition indicator; an ileocecal junction indicator; and an excretion indicator; is identifiable and thus the timing of the corresponding motility event may be determined. Or, the time series may add confidence to a timing determined from, for example, gas sensor data or antenna reflectance data.

An exemplary algorithm for processing the accelerometer sensor data to obtain the representative metric measures a tilt angle between a capsule reference axis or line in fixed relation to the capsule (for example, the capsule long axis), and a reference axis, line, or plane in fixed relation to the earth (for example, a horizontal). The metric is cumulative and increases by an amount that the tilt angle exceeds a hysteresis range. Exceeding the hysteresis range causes the metric to increase and also drags the hysteresis range by the same amount. The metric tracks the cumulative angle travelled by the capsule by reference to a two-dimensional representation. Advantageously, the algorithm filters out roll around the capsule reference axis or line, for example, if the capsule reference axis or line is the long axis of the capsule, the algorithm filters out roll around the long axis (and for this reason may be referred to as capsule tumble). The algorithm is fast and computationally efficient and the metric traces a clear signal.

### Worked Example: Obtaining Time Series Data

FIGS. 9 and 10 each illustrate a respective time series of readings from gas sensor hardware in live trials from ingestible capsule devices ingested by different subjects. Reference or trend lines are illustrated as dashed lines. In particular, the time series is readings from a TCD gas sensor corrected to account for variations in environmental temperature, which variations are measured by an environmental temperature sensor 14a on-board the capsule 10. The subject in FIG. 9 is a True Negative subject (absence of SIBO), as established by current clinical standard aspirate testing, and the subject in FIG. 10 is a True Positive (presence of SIBO). Gastric-duodenal transition events and ileocecal junction transition events are illustrated at FIGS. 9 and 10. The data points are TCD gas sensor readings corrected to compensate for changes in environmental temperature. Readings from the first 30 minutes after gastric duodenal transition timing are cropped out before processing to calculate the trend line and the metric representing fluctuation. The 30 minute value is configurable and values such as 5 minutes or less, between five minutes and 10 minutes, between 10 minutes and 15 minutes, between 15 minutes and 30 minutes, or between 30 minutes and one hour, may also be used. Optionally, such a cropping out could be performed preceding the determined ileocecal junction indicator timing, so that the relevant period begins at or a fixed duration after determined gastric duodenal transition timing and ends at or a fixed duration before determined ileocecal junction transition timing. It is noted that, whilst the capsule 10 is in the small intestine for all of the relevant time period, the relevant time period is not necessarily all of the time in which the capsule 10 is in the small intestine.

### Worked Example: Determine Metric Representing Fluctuation

A first characteristic of the gas sensor data in the relevant time period is calculated at step S20: metric representing fluctuation of concentration of a gas or gases. In the worked example, the metric representing fluctuation is a summation or aggregation of deviation from the reference line as a scalar value. Above a predefined threshold value, the metric representing fluctuation is a fermentation indicator. Which may also be considered to be cumulative magnitude distance between the data points and a trend line or fixed line such as X-axis. Noting that magnitude is considered and not direction, since the characteristic is to quantify variability of the gas sensor data. The on-board processor may be configured to determine the relevant period and calculate the first characteristic, or the gas sensor data may be transmitted by the capsule 10 to a receiver apparatus 30 for processing at the receiver apparatus 30 itself or at a remote computing apparatus 20 to determine the relevant period and calculate the first characteristic.

FIG. 11A illustrates deviation from the baseline but at a low level not indicative of SIBO. The larger area between the data points and reference line in FIG. 11B illustrates greater fermentation and bacterial load and is indicative of SIBO.

FIGS. 11A and 11B each illustrate a respective time series of readings from gas sensor hardware in live trials from ingestible capsule devices ingested by different subjects. Reference or trend lines are illustrated as dashed lines. In particular, the time series is readings from a TCD gas sensor corrected to account for variations in environmental temperature, which variations are measured by an environmental temperature sensor 14a on-board the capsule 10. The subject in FIG. 11A is a True Negative subject (absence of SIBO), as established by current clinical standard aspirate testing, and the subject in FIG. 11B is a True Positive (presence of SIBO). The data points are TCD gas sensor readings corrected to compensate for changes in environmental temperature. Readings from the first 30 minutes after gastric duodenal transition timing are cropped out before processing to calculate the aggregate fluctuation (first characteristic) and trend line gradient (second characteristic). Aggregate fluctuation is summation of magnitude of difference between each data point and a reference line, wherein the reference line may be the x-axis or may be a trend line fitted to the data points. Visually the aggregate fluctuation is identifiable as area under a continuous line joining the data points and the reference line (or specifically area between the said continuous line and the reference line).

In each of FIGS. 11A and 11B the reference line is a trend line fitted to the data points in the relevant period, respectively.

In the example of FIG. 11A the subject is aspirate negative for SIBO and it can be seen that the gas sensor data exhibits little fluctuation from the reference line during the relevant period. Concentrations of gases sensed by the TCD gas sensor are consistent. For completeness, it is noted that the data in FIG. 11A could be a result of different gases varying in concentration but coincidentally cancelling one another out in terms of influence on the thermal conductivity of the gas mixture.

In the example of FIG. 11B the subject is aspirate positive for SIBO and it can be seen that the gas sensor data exhibits significant fluctuations from the trend line during the relevant period. Such fluctuations are caused by varying concentrations of constituent gases to which the gas sensor hardware is exposed during passage through the small intestine. The link to SIBO is based on the hypothesis that the fluctuations are as a consequence of fermentation associated with bacterial overgrowth.

Worked Example: Compare Metric Representing Fluctuation with Threshold(s)

At S30 the metric representing fluctuation, such as aggregate fluctuation, calculated at S20 is compared with a predefined threshold. The threshold is set based on trial data such as illustrated in FIGS. 11A and 11B, with the intention being in setting the threshold that the threshold distinguishes aspirate positive cases from others. It is noted that normalisation according to duration of the time period may be applied. Furthermore, it is noted that there may be two predefined thresholds that do not necessarily coincide: a positive indicator threshold, above which the metric representing fluctuation is indicative of SIBO, and a negative indicator threshold below which the metric representing fluctuation is indicative of SIBO negative (as set out below, the positive threshold may itself be divided into an independent positive threshold and a dependent positive threshold). There may be a gap between the positive and negative indicator thresholds (the negative indicator threshold being lower than the positive indicator threshold) wherein metric representing fluctuation values falling within the gap are not indicative of positive SIBO or negative SIBO. The positive and negative indicator thresholds may coincide.

Furthermore, it is noted that SIBO may be diagnosed based only on the comparison at S30, based on metric representing fluctuation only, or it may be combined with gradient of the trend line at S32. Methods may apply two positive indicator thresholds to metric representing fluctuation at S30: an independent positive indicator threshold above which SIBO is diagnosed without the result of the trend line gradient comparison at S32, and a, lower, dependent positive indicator threshold above which SIBO is diagnosed in dependence also upon the result of the trend line gradient comparison at S32.

Worked Example: Compare Trend Line Gradient with Threshold(s)

At S22 of FIG. 4C, 4D, 4E, 4G a trend line is fitted to the gas sensor data. It is noted that a trend line may also be fitted to the data as part of the metric representing fluctuation calculation at S20 to serve as a reference line. The trend line may be the same in each instance.

At S32 the gradient of the trend line calculated or fitted at S22 is compared with a predefined threshold. Trend line gradient may be referred to as a second characteristic of the gas sensor data. The trend line is a first order polynomial. FIGS. 12A and 12B each illustrates a respective time series of readings from gas sensor hardware in live trials from ingestible capsule devices ingested by different subjects. In particular, the time series is readings from a TCD gas sensor corrected to account for variations in environmental temperature, which variations are measured by an environmental temperature sensor 14a on-board the capsule 10. The threshold is set based on trial data such as illustrated in FIGS. 12A and 12B, with the intention being in setting the threshold that the threshold distinguishes aspirate positive cases from others. Furthermore, it is noted that there may be two predefined thresholds that do not necessarily coincide: a positive indicator threshold, above which the trend line gradient is indicative of SIBO, and a negative indicator threshold below which the trend line gradient is indicative of SIBO negative (as set out below, the positive indicator threshold may itself be divided into an independent positive indicator threshold and a, lower, dependent positive indicator threshold). There may be a gap between the positive and negative indicator thresholds (the negative threshold being lower than the positive threshold) wherein trend line gradient values falling within the gap are not indicative of positive SIBO or negative SIBO. The positive and negative indicator thresholds may coincide.

Furthermore, it is noted that SIBO may be diagnosed based only on the comparison at S30, based only on the comparison at S32, or based on a combination of the comparisons at S30 and S32. Methods may apply two positive thresholds to trend line gradients at S32: an independent positive threshold above which SIBO is diagnosed without the result of the metric representing fluctuation comparison at S32, and a, lower, dependent positive threshold above which SIBO is diagnosed in dependence also upon the result of the trend line gradient comparison at S32.

The ingestible capsule devices 10 used in the trials and data gathering exercises are designed and produced by Atmo Biosciences Pty Ltd and may be referred to as Atmo gas capsule.

### Trial Data: ROC and Thresholding

FIGS. 13A and 13B illustrate sensitivity against specificity for trend line gradient (FIG. 13A) and aggregate fluctuation or area under curve AUC (FIG. 13B). The ROC, Receiver Operating Characteristic, curves of FIGS. 13A and 13B use clinical aspirate testing as the only source of truth. Thresholds for trend line gradient and aggregate fluctuation are selectable based on the ROC curves in accordance with desired selectivity and specificity. Thresholds may be configured to be combined to make positive determinations of SIBO presence (referred to elsewhere as dependent positive thresholds). More detail on combining measures is provided below. Thresholds may be selected as standalone thresholds so that presence of SIBO is determined based on either aggregate fluctuation or trend line gradient alone (independent positive thresholds).

### Trial Data: Combining Aggregate Fluctuation and Trend Line Gradient

The independent positive thresholds for trend line gradient and aggregate fluctuation may be applied to make a determination of whether or not SIBO is present in a subject based on either characteristic alone. Alternatively, the two thresholds may be combined, so that for SIBO to be determined as present in a subject both the trend line gradient and the aggregate fluctuation, AUC, must meet respective gradients. FIGS. 4C and 4D illustrate such methods. Combining the two characteristics in this way improves confidence in the determination.

FIGS. 14A to 14C each illustrates a respective time series of readings from gas sensor hardware in live trials from ingestible capsule devices ingested by different subjects. In particular, the time series is readings from a TCD gas sensor corrected to account for variations in environmental temperature, which variations are measured by an environmental temperature sensor 14a on-board the capsule 10.

FIG. 14A illustrates gas sensor data (temperature corrected TCD) from a trial in which the trend line gradient does not exceed the threshold set for trend line gradient, and the aggregate fluctuation does not meet the threshold set for aggregate fluctuation, so the determination is that SIBO is absent.

FIG. 14B illustrates gas sensor data (temperature corrected TCD) from a trial in which the trend line gradient does exceed the threshold set for trend line gradient, but the aggregate fluctuation does not meet the threshold set for aggregate fluctuation, so the determination is that SIBO is absent.

FIG. 14C illustrates gas sensor data (temperature corrected TCD) from a trial in which the trend line gradient does exceed the threshold set for trend line gradient, and the aggregate fluctuation meets the threshold set for aggregate fluctuation, so the determination is that SIBO is present.

FIGS. 15A and 15B illustrate gas sensor data (temperature corrected TCD) from further trials in which the trend line gradient does exceed the threshold set for trend line gradient, and the aggregate fluctuation meets the threshold set for aggregate fluctuation, so the determination is that SIBO is present. This is in agreement with jejunal aspirate results for the same subjects.

FIGS. 16A and 16B illustrate gas sensor data (temperature corrected TCD) from further trials in which the aggregate fluctuation does not meet the threshold set for aggregate fluctuation, so the determination is that SIBO is absent, based on the combination of thresholds test. This is in agreement with jejunal aspirate results for the same subjects.

FIG. 17 illustrates results of comparison between the present two-threshold technique (referred to as Atmo Dx Method) and jejunal aspirate testing, from 23 trials (i.e. 23 capsules given to 23 different subjects each with respective jejunal aspirate positive negative results). The method is 83% accurate taking jejunal aspirate as truth. It is noted that jejunal aspirate testing suffers from inaccuracies and that therefore the accuracy of agreement with jejunal aspirate testing is not necessarily the true accuracy, which may be higher or lower.

FIG. 18 illustrates a hardware arrangement of an apparatus configured to perform a method or methods described in this specification. FIG. 18 is a schematic illustration of a hardware arrangement of a computing apparatus. The methods described herein may be performed by apparatus having an arrangement such as illustrated in FIG. 18. Apparatus having processor hardware and memory hardware described in the present specification may include one or more devices having an arrangement such as illustrated in FIG. 18. A plurality of such devices may be interconnected over a network such as a Local Area Network or the internet. A cloud service including performing one or more of the methods described in the present specification may be performed by one or more devices having an arrangement such as illustrated in FIG. 18.

The computing apparatus comprises a plurality of components interconnected by a bus connection. The bus connection is an exemplary form of data and/or power connection. Direct connections between components for transfer of power and/or data may be provided in addition or as alternative to the bus connection.

The computing apparatus comprises memory hardware 991 and processing hardware 993, which components are essential regardless of implementation. Further components are context-dependent, including a network interface 995, input devices 997, and a display unit 999. The display unit 999 and the processing hardware 993 may cooperate to implement a graphical user interface.

The memory hardware 991 stores processing instructions for execution by the processing hardware 993. The memory hardware 991 may include volatile and/or non-volatile memory. The memory hardware 991 may store data pending processing by the processing hardware 993 and may store data resulting from processing by the processing hardware 993.

The processing hardware 993 comprises one or a plurality of interconnected and cooperative CPUs for processing data according to processing instructions stored by the memory hardware 991.

A computing apparatus may comprise one computing device according to the hardware arrangement of FIG. 18, or a plurality of such devices operating in cooperation with one another. For example, in a client:server arrangement.

A network interface 995 provides an interface for transmitting and receiving data over a network. Connectivity to one or more networks is provided. For example, a local area network and/or the internet. Connectivity may be wired and/or wireless.

Input devices 997 provide a mechanism to receive inputs from a user. For example, such devices may include one or more from among a mouse, a touchpad, a keyboard, an eye-gaze system, and a touch interface of a touchscreen. Inputs may be received over a network connection. For example, in the case of server computers, a user may connect to the server over a connection to another computing apparatus and provide inputs to the server using the input devices of the another computing apparatus.

A display unit 999 provides a mechanism to display data visually to a user. The display unit 999 may display user interfaces by which certain locations of the display unit become functional as buttons or other means allowing for interaction with data via an input mechanism such as a mouse. A server may connect to a display unit 999 over a network.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

1.-37. (canceled)

38. An ingestible capsule device comprising:
an ingestible indigestible bio-compatible housing; and,
within the housing:
a power source;
sensor hardware including gas sensor hardware;
processor hardware;
memory hardware; and
a wireless data transmitter;
the memory hardware storing processing instructions which, when executed by the processor hardware, cause the processor hardware to perform a process for detecting presence or absence of small-intestinal bacterial overgrowth, SIBO, in a subject, the process comprising:
obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of composition of the gas mixture at the location of the ingestible capsule device in the gastrointestinal tract of the subject;
using the gas sensor data to calculate a metric representing fluctuation of the gas sensor data during a passage of the ingestible capsule device through the small intestine of the gastrointestinal tract;
determining presence or absence of SIBO in the subject at least partially in dependence upon the metric representing fluctuation.

39. The ingestible capsule device according to claim 38, wherein the determining comprises, as a first comparison, comparing the metric representing fluctuation with a predefined threshold, and using a result of the first comparison to determine presence or absence of SIBO, and wherein the metric representing fluctuation is an aggregate fluctuation such as a cumulative aggregate fluctuation, or wherein the metric representing fluctuation is a standard deviation or variance from a trend line.

40. The ingestible capsule device according to claim 38, wherein the gas sensor data is obtained by obtaining readings from an environmental temperature sensor housed within the ingestible capsule device representing environmental temperature at the ingestible capsule device, and compensating sampled values of an output signal generated by the gas sensor hardware to account for variations in environmental temperature, the gas sensor data being the compensated values.

41. The ingestible capsule device according to claim 38, wherein the gas sensor data represents a concentration of a specific gas or gases;
wherein the gas sensor data is obtained by processing sampled values of an output signal generated by the gas sensor hardware to extract the concentration of the specific gas or gases; and
wherein the specific gas or gases is one or more from among: carbon dioxide CO2, hydrogen H2, methane, and one or more VOCs.

42. The ingestible capsule device according to claim 41, wherein determining presence or absence of SIBO in the subject at least partially in dependence upon the concentration of the specific gas or gases exceeding a predefined threshold concentration at one or a predefined threshold number of locations during passage of the ingestible capsule device through the small intestine; and
wherein the gas sensor data is, or is in direct proportion to, sampled values of an output signal generated by the gas sensor hardware.

43. The ingestible capsule device according to claim 38, wherein the process further comprises:
fitting a trend line to the gas sensor data;
wherein the determining presence or absence of SIBO in the subject is at least partially in dependence upon the metric representing fluctuation and at least partially in dependence upon a gradient of the trend line or an average gradient of the trend line.

44. The ingestible capsule device according to claim 38, wherein the determining comprises, as a second comparison, comparing a gradient of the trend line with a second predefined threshold;
combining a result of the first comparison the gradient with the result of the second comparison to detect presence or absence of small-intestinal bacterial overgrowth in the subject; and
wherein
the determining comprises calculating a weighted average or weighted sum of characteristics including at least the metric representing fluctuation and the trend line gradient, comparing the weighted average with a predefined threshold, and determining presence or absence of SIBO in dependence upon the result of the comparison; and
wherein the gas sensor data represents a concentration of a specific gas or gases, and the characteristics further comprise a number of times, or a duration for which, during passage of the capsule through the small intestine that the concentration of the specific gas or gases exceeds a predefined threshold concentration.

45. The ingestible capsule device according to claim 38, wherein the gas sensor hardware comprises a TCD gas sensor and the gas sensor data represents a time series of readings from the TCD gas sensor.

46. The ingestible capsule device according to claim 38, wherein the process further comprises at least one of:
(i) detecting a gas sensor data gastric-duodenal transition indicator among the gas sensor data and/or detecting a gas sensor data ileocecal junction transition indicator among the gas sensor data, and based on a timing of the detected gas sensor data gastric-duodenal transition indicator and/or the detected gas sensor data ileocecal junction transition indicator, determining timing of the passage of the ingestible capsule device through the small intestine of the subject;
(ii) obtaining accelerometer data representing a time series of readings from an accelerometer housed within the ingestible capsule device, the time series of readings being taken during the passage of the ingestible capsule device through the gastrointestinal tract of the subject; and detecting an accelerometer data gastric-duodenal indicator and/or an accelerometer data ileocecal junction indicator in the accelerometer data, and determining the timing of the passage of the ingestible capsule device through the small intestine of the gastrointestinal tract based on the accelerometer data gastric-duodenal indicator and/or the accelerometer data ileocecal junction indicator; and
(iii) obtaining reflectometer data representing a time series of readings from a reflectometer housed within the ingestible capsule device, the reflectometer comprising a transmission antenna connected in series with a directional coupler configured to measure a reflected signal from the transmission antenna, the time series of readings being taken during the passage of the ingestible capsule device through the gastrointestinal tract of the subject; and detecting a reflectometer data gastric-duodenal indicator and/or a reflectometer data ileocecal junction indicator in the reflectometer data, and determining the timing of the passage of the ingestible capsule device through the small intestine of the gastrointestinal tract based on the reflectometer data gastric-duodenal indicator and/or the reflectometer ileocecal junction indicator.

47. The ingestible capsule device according to claim 46, wherein:
determining the timing of the passage of the ingestible capsule device through the small intestine of the subject comprises: determining a timing of a passage of the ingestible capsule device across the gastric-duodenal junction based on one or more from among: the gas sensor data gastric-duodenal indicator; the accelerometer data gastric-duodenal indicator; and the reflectometer data gastric-duodenal indicator.

48. The ingestible capsule device according to claim 47, wherein determining the timing of the passage of the ingestible capsule device through the small intestine of the subject comprises:
determining a timing of a passage of the ingestible capsule device across the ileocecal junction based on one or more from among: the gas sensor data ileocecal junction indicator; the accelerometer data ileocecal junction indicator; and the reflectometer data ileocecal junction indicator.

49. The ingestible capsule device according to claim 38, the process further comprising:
quantifying an amount of small-intestinal bacterial overgrowth in the subject according to the value of the metric representing fluctuation,
quantifying an amount of small-intestinal bacterial overgrowth in the subject according to the gradient of the trend line, or
quantifying an amount of small-intestinal bacterial overgrowth in the subject according to a number of times, or a duration for which, during passage of the capsule through the small intestine that the concentration of the specific gas or gases exceeds a predefined threshold concentration.

50. The ingestible capsule device according to claim 38, the process further comprising:
generating a report including the detected presence or absence of small-intestinal bacterial overgrowth in the subject.

51. The ingestible capsule device according to claim 50, further comprising:
based on one or more from among:
a detected fermentation indicator,
the metric representing fluctuation,
a number of events, or a duration for which, during passage of the capsule through the small intestine that a concentration of the specific gas or gases represented by the gas sensor data exceeds a predefined threshold concentration, and
the gradient of a trend line fitted to the gas sensor data,
measuring a level of fermentation activity detected in the small intestine of the subject, and including the measured level in the generated report.

52. The ingestible capsule device according to claim 50, the process further comprising determining, based on a timing of:
deviations from a trend line contributing to a metric representing fluctuation, and/or a timing of events at which concentration of a specific gas or gases represented by the gas sensor data exceeds a predefined threshold concentration;
an estimated location or locations within the small intestine of fermentation activity; wherein
the report further comprises the measured level of fermentation activity and/or the estimated location or locations within the small intestine of fermentation activity.

53. The ingestible capsule device according to any of claim 50, wherein
the process further comprises wirelessly transmitting the report to a receiver device outside of the body of the subject.

54. The ingestible capsule device according to claim 53, wherein
the wirelessly transmitting is via a Bluetooth transceiver housed by the ingestible capsule device.

55. A non-transitory computer-readable medium storing processing instructions which, when executed by processor hardware, causes the processor hardware to perform a process comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of a composition of the gas mixture at the location of the ingestible capsule device in the gastrointestinal tract of the subject; using the gas sensor data to calculate a gradient of a first order polynomial best fit line fitted to the gas sensor data from passage of the ingestible capsule device through the small intestine of the gastrointestinal tract; and determining presence or absence of SIBO in the subject at least partially in dependence upon the gradient of the best fit line.

56. A non-transitory computer-readable medium storing processing instructions which, when executed by processor hardware, causes the processor hardware to perform a process comprising: obtaining gas sensor data representing a time series of readings from gas sensor hardware housed within an ingestible capsule device orally ingested by the subject, the time series of readings being taken during exposure of the gas sensor hardware to a gas mixture at the ingestible capsule device during passage of the ingestible capsule device through a gastrointestinal tract of the subject, the gas sensor hardware being sensitive to changes of a composition of the gas mixture at the location of the ingestible capsule device in the gastrointestinal tract of the subject;
wherein the gas sensor data represents a concentration of a specific gas or gases;
the method further comprising determining presence or absence of SIBO in the subject at least partially in dependence upon the concentration of the specific gas or gases exceeding a predefined threshold concentration at one or a predefined threshold minimum number of locations during passage of the ingestible capsule device through the small intestine.

57. The non-transitory computer-readable medium according to claim 56, wherein the specific gas or gases is one or more from among hydrogen, carbon dioxide, and methane.
